(19) United States
(12) Patent Application Publication
(10) Pub. No.: US 2025/0354932 A1
(43) Pub. Date: Nov. 20, 2025

(54) IMAGING DEVICE, OPTICAL COMPONENT, AND MEASUREMENT SYSTEM

(71) Applicant: Panasonic Intellectual Property Management Co., Ltd., Osaka (JP)

(72) Inventor: KATSUYA NOZAWA, Osaka (JP)

(21) Appl. No.: 19/287,901

(22) Filed: Aug. 1, 2025

Related U.S. Application Data
(63) Continuation of application No. PCT/JP2024/002731, filed on Jan. 30, 2024.

(30) Foreign Application Priority Data
Feb. 27, 2023 (JP) 2023-028528
Feb. 27, 2023 (JP) 2023-028529
Mar. 31, 2023 (JP) 2023-058148
Dec. 8, 2023 (JP) 2023-208038

(51) Int. Cl. G01N 21/64

(52) U.S. Cl. CPC G01N 21/6456; G01N 2021/6463; G01N 2201/0636; G01N 2201/0638; G01N 2201/0683

(57) ABSTRACT

An imaging device includes a first optical element that separates a light beam from a subject into a first light beam and a second light beam having optical characteristics different from those of the first light beam, an imaging optical system on which the first light beam and the second light beam are incident at different angles from each other, the imaging optical system forming a first image by imaging the first light beam and forming a second image by imaging the second light beam, and an image sensor including an imaging surface. The first image and the second image are formed at different positions on the imaging surface. The first image and the second image are formed symmetrically on the imaging surface with respect to a plane that intersects the imaging surface.

### BACKGROUND

### 1. Technical Field

The present disclosure relates to an imaging device, an optical component, and a measurement system.

### 2. Description of the Related Art

Obtaining two images of a subject with different optical characteristics is useful for evaluating the subject. For example, obtaining two images of a subject with wavelengths in different wavelength ranges is useful for two-color thermography to estimate the temperature of the subject. The principle of two-color thermography is as follows. The intensity of thermal radiation from an object depends on the temperature and emissivity of the object. Therefore, if the emissivity is unknown, the temperature of the object cannot be determined by measuring only the intensity of thermal radiation from the object. However, if the emissivities of two different wavelength ranges can be treated as equal, the ratio of the radiation intensities of these two wavelength ranges depends on the temperature but not on the emissivity. In that case, the temperature of the object can be estimated by measuring the radiation intensities of these two wavelength ranges from the same point in the subject and calculating the intensity ratio.

Obtaining two images of a subject in different wavelength ranges is also useful for fluorescence imaging. In fluorescence imaging, the fluorescence intensity is proportional to the excitation light intensity and luminous efficiency. Therefore, by imaging the fluorescence intensity and the excitation light intensity and calculating the intensity ratio thereof, the distribution of luminous efficiency can be visualized.

Japanese Unexamined Patent Application Publication No. 2002-214048 and Japanese Unexamined Patent Application Publication No. 55-124379 disclose examples of an imaging device that obtains two images of a subject in different wavelength ranges.

### SUMMARY

In one general aspect, the techniques disclosed here feature an imaging device including: a first optical element that separates a light beam from a subject into a first light beam and a second light beam having optical characteristics different from optical characteristics of the first light beam; an imaging optical system on which the first light beam and the second light beam are incident at different angles from each other, the imaging optical system forming a first image by imaging the first light beam and forming a second image by imaging the second light beam; and an image sensor including an imaging surface. The first image and the second image are formed at different positions on the imaging surface. The first image and the second image are formed symmetrically on the imaging surface with respect to a plane that intersects the imaging surface.

General or specific aspects of the present disclosure may be realized as a system, a device, a method, an integrated circuit, a computer program, a recording medium such as a computer-readable recording disk, or any given combination thereof. The computer-readable recording medium may include a non-volatile recording medium such as a CD-ROM (Compact Disc-Read Only Memory). A device may include one or more devices. When a device includes two or more devices, the two or more devices may be arranged in one apparatus, or may be arranged separately in two or more separate apparatuses. In this specification and the claims, the term “device” may mean not only one device, but also a system consisting of a plurality of devices.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram schematically illustrating a configuration of an imaging device according to exemplary Embodiment 1 of the present disclosure;

FIG. 2 is a diagram schematically illustrating how a light beam is reflected by a mirror surface;

FIG. 3 is a diagram schematically illustrating the configuration of the imaging device according to exemplary Embodiment 1 of the present disclosure;

FIG. 4 is a diagram schematically illustrating an example of a dichroic prism;

FIG. 5 is a diagram schematically illustrating an example of optical paths of light beams within the dichroic prism;

FIG. 6 is a diagram schematically illustrating an example of an optical path adjusting element;

FIG. 7 is a diagram schematically illustrating an example of a path of a light beam within the optical path adjusting element;

FIG. 8 is a diagram schematically illustrating paths of light beams within the optical component;

FIG. 9 is a diagram for explaining general characteristics of an imaging optical system;

FIG. 10 is a diagram schematically illustrating an example of the paths of light beams within the imaging device;

FIG. 11 is a diagram schematically illustrating how a principal ray emitted from a certain point is imaged on an image sensor when no dichroic prism is provided;

FIG. 12A is a diagram for explaining an intermediate apparent position of the subject for a first image;

FIG. 12B is a diagram for explaining an actual position of the subject for the first image;

FIG. 13 is a diagram for explaining an actual position of the subject for a second image;

FIG. 14 is a diagram schematically illustrating how the first light beam travels through a first Littrow prism;

FIG. 15A is a diagram schematically illustrating how a light ray emitted from a certain point is imaged on the image sensor for the first light beam;

FIG. 15B is another diagram schematically illustrating how a light ray emitted from a certain point is imaged on the image sensor for the first light beam;

FIG. 16 is a diagram schematically illustrating how the second light beam travels through a second Littrow prism;

FIG. 17A is a diagram schematically illustrating how a light ray emitted from a certain point is imaged on the image sensor for the second light beam;

FIG. 17B is another diagram schematically illustrating how a light ray emitted from a certain point is imaged on the image sensor for the second light beam;

FIG. 18 is a diagram schematically illustrating a range in which the first and second light beams are imaged on the image sensor;

FIG. 19 is a diagram for explaining a positional relationship between an apparent subject and an actual subject;

FIG. 20 is a diagram schematically illustrating an example of first and second images formed on the image sensor;

FIG. 21 is a diagram schematically illustrating how light rays emitted from a certain point are imaged on the image sensor for the first and second light beams;

FIG. 22 is a diagram schematically illustrating a positional relationship between an apparent light shielding body, an actual light shielding body, and a dichroic prism;

FIG. 23 is a diagram schematically illustrating a range of blocking the optical path along which the first and second light beams are both imaged on the image sensor;

FIG. 24 is a diagram schematically illustrating an example of a filter array;

FIG. 25A is a diagram schematically illustrating an example of connecting an optical component and a lens unit;

FIG. 25B is a diagram schematically illustrating another example of connecting the optical component and the lens unit;

FIG. 25C is a diagram schematically illustrating yet another example of connecting the optical component and the lens unit;

FIG. 26 is a diagram schematically illustrating a specific configuration of an optical component in Modification 1 of the imaging device according to Embodiment 1;

FIG. 27 is a diagram schematically illustrating an optical element group in Modification 1 of the imaging device according to Embodiment 1;

FIG. 28 is a diagram schematically illustrating an example of paths of light beams within the optical element group in Modification 1 of the imaging device according to Embodiment 1;

FIG. 29 is a diagram schematically illustrating an optical path adjusting element in Modification 1 of the imaging device according to Embodiment 1;

FIG. 30 is a diagram schematically illustrating an example of a path of a light beam within the optical path adjusting element in Modification 1 of the imaging device according to Embodiment 1;

FIG. 31 is a diagram schematically illustrating an example of paths of light beams within the optical component in Modification 1 of the imaging device according to Embodiment 1;

FIG. 32 is a diagram schematically illustrating an example of the paths of the light beams in Modification 1 of the imaging device according to Embodiment 1;

FIG. 33 is a diagram schematically illustrating an example of a specific configuration of an optical component in Modification 2 of the imaging device according to Embodiment 1;

FIG. 34 is a diagram schematically illustrating an example of a dichroic prism in Modification 2 of the imaging device according to Embodiment 1;

FIG. 35 is a diagram schematically illustrating an example of paths of light beams within the dichroic prism in Modification 2 of the imaging device according to Embodiment 1;

FIG. 36 is a diagram schematically illustrating an example of paths of light beams within the optical component in Modification 2 of the imaging device according to Embodiment 1;

FIG. 37 is a diagram schematically illustrating an example of paths of light beams in Modification 2 of the imaging device according to Embodiment 1;

FIG. 38 is a diagram schematically illustrating a specific configuration of an imaging device according to exemplary Embodiment 2 of the present disclosure;

FIG. 39 is a diagram schematically illustrating a configuration of Modification 1 of the imaging device according to Embodiment 2;

FIG. 40 is a diagram schematically illustrating a configuration of Modification 2 of the imaging device according to Embodiment 2;

FIG. 41 is a diagram schematically illustrating a configuration of Modification 3 of the imaging device according to Embodiment 2;

FIG. 42A is a diagram schematically illustrating an example of a measurement system including the imaging device according to Embodiment 1;

FIG. 42B is a diagram schematically illustrating an example of spectra of thermal radiation from the subject;

FIG. 43A is a diagram schematically illustrating another example of a measurement system including the imaging device according to Embodiment 1;

FIG. 43B is a diagram schematically illustrating an example of spectra of excitation light and fluorescence;

FIG. 44A is a diagram schematically illustrating yet another example of a measurement system including the imaging device according to Embodiment 1;

FIG. 44B is a diagram schematically illustrating an example of an absorption spectrum of water;

FIG. 44C is a diagram schematically illustrating an example of a spectrum of illuminating light;

FIG. 45 is a diagram schematically illustrating Example 1 of an imaging device of the related art based on Japanese Unexamined Patent Application Publication No. 2002-214048;

FIG. 46 is a diagram schematically illustrating an example of images in the first and second wavelength ranges formed on the image sensor;

FIG. 47A is a diagram schematically illustrating an example of curvature aberration;

FIG. 47B is a diagram schematically illustrating another example of curvature aberration; and

FIG. 48 is a diagram schematically illustrating Example 2 of an imaging device of the related art based on Japanese Unexamined Patent Application Publication No. 55-124379.

### DETAILED DESCRIPTIONS

The present disclosure provides an imaging device capable of obtaining, with a simple configuration, two images having different optical characteristics suitable for evaluating a subject.

In the present disclosure, all or part of a circuit, unit, device, member, or part, or all or some of functional blocks in a block diagram, may be implemented by one or more electronic circuits including, for example, a semiconductor device, a semiconductor integrated circuit (IC), or an LSI (large scale integration). The LSI or IC may be integrated into one chip, or may be configured by combining a plurality of chips. For example, functional blocks other than memory elements may be integrated into one chip. Such circuits are referred to here as the LSI or IC, but may also be referred to differently as a system LSI, VLSI (very large scale integration) or ULSI (ultra large scale integration) depending on the degree of integration. A field programmable gate array (FPGA), which is programmed after the LSI is manufactured, or a reconfigurable logic device that can reconfigure the junction relationships within the LSI or set up circuit partitions within the LSI, can also be used for the same purpose.

Furthermore, all or some of the functions or operations of the circuit, unit, device, member, or part can be executed by software processing. In this case, the software is recorded in one or more non-transitory recording media such as a ROM, an optical disk, and a hard disk drive. When the software is executed by a processor, functions specified by the software are executed by the processor and peripheral devices. The system or device may include one or more non-transitory recording media storing the software, a processor, and necessary hardware devices, such as interfaces.

In the present disclosure, “light” means electromagnetic waves including not only visible light with a wavelength of about 400 nm to about 700 nm, but also ultraviolet light with a wavelength of about 10 nm to about 400 nm and infrared light with a wavelength of about 700 nm to about 1 mm.

Exemplary embodiments of the present disclosure will be described below. Note that the embodiments described below are all comprehensive or specific examples. The numerical values, shapes, components, arrangement and connection of the components, steps, and the order of steps described in the following embodiments are merely examples and are not intended to limit the present disclosure. Furthermore, among the components in the following embodiments, components that are not described in the independent claims indicating the highest concept are described as optional components. The drawings are schematic and are not necessarily drawn to scale. Furthermore, in the drawings, substantially the same components are denoted by the same reference numerals, and redundant description thereof may be omitted or simplified.

Prior to description of the embodiments of the present disclosure, the underlying knowledge forming the basis of the present disclosure will be described below. Examples 1 and 2 of an imaging device according to the related art that obtain two images of a subject in different wavelength ranges will be described. The two images in different wavelength ranges are an example of two images having different optical characteristics. Example 1 of Imaging Device of the Related Art

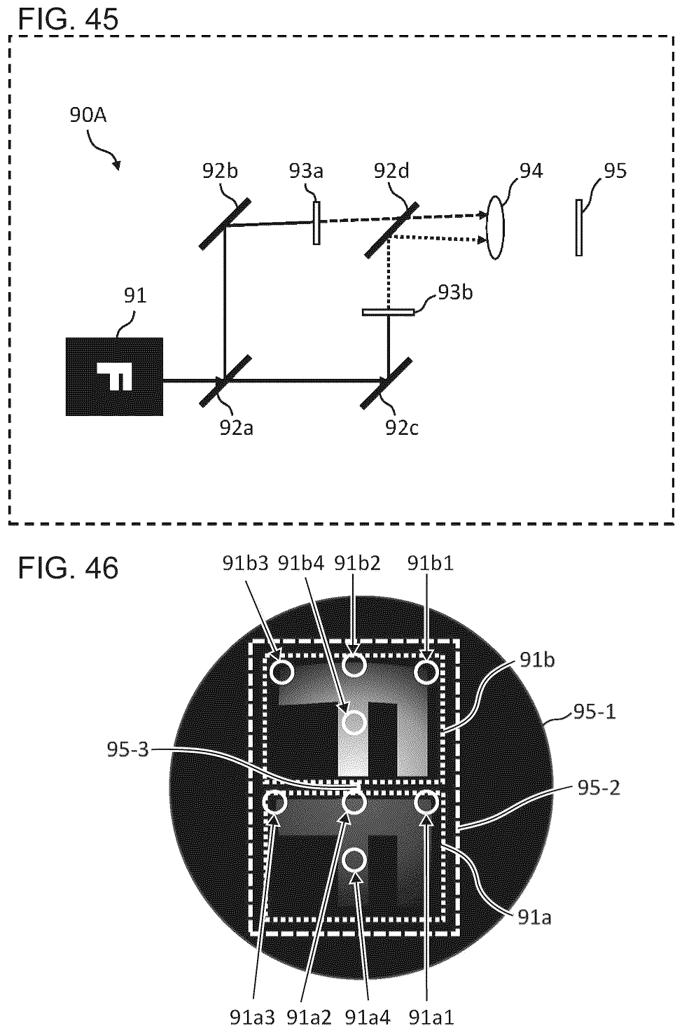

FIG. 45 is a diagram schematically illustrating Example 1 of an imaging device of the related art based on Japanese Unexamined Patent Application Publication No. 2002-214048. In an imaging device 90A illustrated in FIG. 45, a light beam from a subject 91 is separated into two light beams by a half mirror 92a. One light beam is reflected by a mirror 92b and passes through a bandpass filter 93a that selectively transmits light having a wavelength included in a first wavelength range. The other light beam is reflected by a mirror 92c and passes through a bandpass filter 93b that selectively transmits light having a wavelength included in a second wavelength range. The dashed arrow in FIG. 45 represents the light beam having the wavelength included in the first wavelength range, and the dotted arrow in FIG. 45 represents the light beam having the wavelength included in the second wavelength range. A half mirror 92d causes these two light beams to enter an imaging optical system 94. The imaging optical system 94 forms images of the light beams having the wavelengths included in the first and second wavelength ranges on an image sensor 95.

The half mirror 92a and the half mirror 92d are arranged at a 45° angle with respect to an optical axis of the imaging optical system 94. On the other hand, the mirror 92b and the mirror 92c are arranged so that images of the first and second wavelength ranges formed by the above two light beams are formed at different positions on an imaging surface of the image sensor 95. When the images of the first and second wavelength ranges do not overlap, the intensity of each image can be easily determined.

FIG. 46 is a diagram schematically illustrating an example of the images of the first and second wavelength ranges formed on the image sensor 95. The subject 91 illustrated in FIG. 45 has a part that has the shape of the letter F and emits light having the wavelengths included in the first and second wavelength ranges on a background that emits almost no light.

Note that the image formed by the imaging optical system 94 is rotated 180° from the subject. However, to facilitate understanding of the relationship with the subject, FIG. 46 illustrates an image rotated to have the same orientation as the subject.

The range of the image formed on the image sensor 95 by the imaging optical system 94 is a circular range called an image circle 95-1, as illustrated in FIG. 46. The image circle 95-1 is set to be wider than an imaging range 95-2 of the image sensor 95. By forming a first image 91a of the first wavelength range and a second image 91b of the second wavelength range within the imaging range 95-2, the radiation intensities of the first and second wavelength ranges can be measured at the same time.

As illustrated in FIG. 46, a point 91b1 in the second image 91b corresponds to a point 91al in the first image 91a. Similarly, a point 91b2 in the second image 91b corresponds to a point 91a2 in the first image 91a. A point 91b3 in the second image 91b corresponds to a point 91a3 in the first image 91a. A point 91b4 in the second image 91b corresponds to a point 91a4 in the first image 91a.

As illustrated in FIG. 46, the first image 91a and the second image 91b formed on the image sensor 95 have the same orientation. This is because the light beams having the wavelengths included in the first and second wavelength ranges are both reflected twice, the same even number of times.

The first image 91a and the second image 91b are in a translation relationship. For this reason, it is impossible to make the distance from a reference position 95-3 consistent for each point corresponding to the same position in the subject 91. The reference position 95-3 is the position where the optical axis of the imaging optical system 94 passes through the imaging range 95-2.

The characteristics of the image formed by the imaging optical system 94 vary depending on the distance from the reference position 95-3, resulting in the following problems.

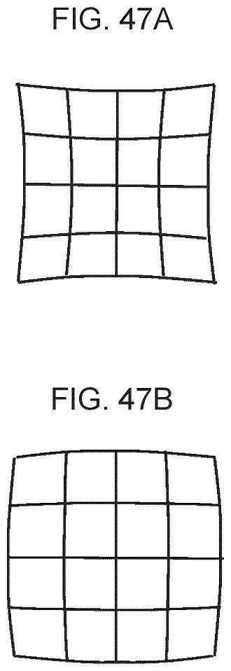

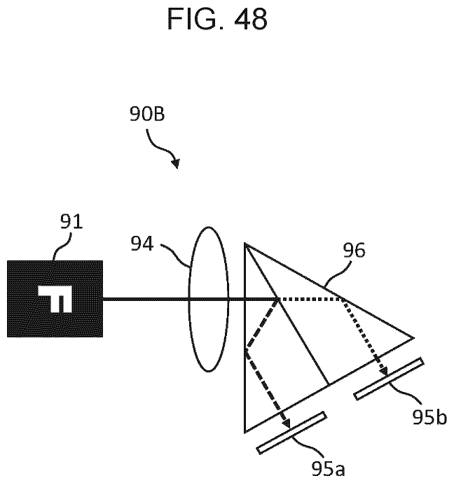

The first problem is curvature aberration. The image formed by the imaging optical system 94 does not generally have a correct similarity relationship with the subject 91. FIGS. 47A and 47B are diagrams schematically illustrating examples of the curvature aberration. Even if a square lattice is formed as an image of the subject 91, the formed square lattice may be distorted like a pincushion shape as illustrated in FIG. 47A or a barrel shape as illustrated in FIG. 47B. The amount of this distortion depends on the relative position with respect to the reference position 95-3.

As illustrated in FIG. 46, the line connecting the points 91a1, 91a2, and 91a3 in the first image 91a is almost straight, but the line connecting the points 91b1, 91b2, and 91b3 in the second image 91b corresponding to the same range is curved. Here, a barrel distortion is illustrated as an example.

When such curvature aberration occurs, the corresponding positional relationship between the first image 91a and the second image 91b is expressed using a complex function. Furthermore, the characteristics of the curvature aberration differ for each imaging optical system 94. Therefore, it is not easy to identify the first position in the first image 91a and the second position in the second image 91b, which correspond to the same position in the subject 91, and it is not easy to calculate the intensity ratio therebetween.

The second problem is peripheral darkening. In general, the image becomes darker as it moves away from the reference position 95-3. This phenomenon is called peripheral darkening. Due to this peripheral darkening, the ratio of the intensity at the first position in the first image 91a to the intensity at the second position in the second image 91b is the ratio of the radiation intensity in the first wavelength range to the radiation intensity in the second wavelength range multiplied by the ratio of the degrees of peripheral darkening. For example, the distance of the point 91b2 from the reference position 95-3 is longer than the distance of the point 91a2 from the reference position 95-3, resulting in a large ratio of the degree of peripheral darkening between the point 91b2 and the point 91a2. On the other hand, the distance of the point 91a4 from the reference position 95-3 is equal to the distance of the point 91b4 from the reference position 95-3, resulting in that the ratio of the degree of peripheral darkening between the point 91b4 and the point 91a4 is approximately 1.

To determine the temperature of the subject 91, it is necessary to calculate the ratio between the intensity of thermal radiation in the first wavelength range and the intensity of thermal radiation in the second wavelength range. However, in the imaging device 90A described in Japanese Unexamined Patent Application Publication No. 2002-214048, the intensity ratio of the first image 91a and the second image 91b formed on the image sensor 95 depends not only on the intensity ratio of thermal radiation but also on the ratio of the degree of peripheral darkening. Furthermore, the ratio of the degree of peripheral darkening differs depending on the positions in the first image 91a and the second image 91b. The ratio of the degree of peripheral darkening also depends on a lens aperture, for example.

Therefore, even if the position in the first image 91a and the position in the second image 91b corresponding to the same position in the subject 91 can be identified, the intensity of thermal radiation in the first and second wavelength ranges cannot be easily determined from the imaging result. This makes it difficult to accurately measure the temperature of the subject 91. The intensity ratio between the first and second wavelength ranges should depend on the thermal radiation and not on the imaging optical system 94. The above problem occurs in all applications that require measurement of the intensity ratio between the first and second wavelength ranges.

### Example 2 of Imaging Device of the Related Art

FIG. 48 is a diagram schematically illustrating Example 2 of an imaging device of the related art based on Japanese Unexamined Patent Application Publication No. 55-124379. In an imaging device 90B illustrated in FIG. 48, a light beam from a subject 91 enters an imaging optical system 94. A dichroic prism 96 separates the light beam that has passed through the imaging optical system 94 into a light beam having a wavelength included in a first wavelength range and a light beam having a wavelength included in a second wavelength range. The dichroic prism 96 then causes the light beam having the wavelength included in the first wavelength range to enter a first image sensor 95a and the light beam having the wavelength included in the second wavelength range to enter a second image sensor 95b. Specifically, the imaging optical system 94 forms an image of the light beam having the wavelength included in the first wavelength range on the first image sensor 95a via the dichroic prism 96. Similarly, the imaging optical system 94 forms an image of the light beam having the wavelength included in the second wavelength range on the second image sensor 95b via the dichroic prism 96.

In the imaging device 90B, the first image sensor 95a and the second image sensor 95b need to be arranged behind the dichroic prism 96. It is also necessary to provide two control circuits to control these two image sensors 95a and 95b. The need for two image sensors and two control circuits is costly. Furthermore, the imaging device 90B cannot perform imaging using a general camera equipped with one image sensor 95. Therefore, it is necessary to spend time and money to develop a camera dedicated to the imaging device 90B.

The imaging device 90B also has limitations on the selection of the imaging optical system 94. A lens unit including the imaging optical system 94 and a camera including the image sensor 95 are generally designed and manufactured based on standards. The lens unit and the camera can be freely combined and used within the scope of the same standard. In the general standard, the joint of the lens unit and the camera has a specified shape. Furthermore, the distance from the joint to the image sensor 95 in the camera, that is, a flange back is also specified. For example, in the C-mount standard, which is widely used for industrial purposes, the joint is specified to have an inner diameter of 25.4 mm, a thread pitch of 0.794 mm, and a flange back of 17.526 mm.

Although the distance from the joint to the rearmost lens surface of the imaging optical system 94 is not usually specified in the standard, this distance is generally allowed to be zero. Therefore, the minimum value of the back focus, which is the distance from the rearmost lens surface of the imaging optical system 94 to the image sensor 95, is generally the value of the flange back.

In the imaging device 90B, the dichroic prism 96 is disposed between the imaging optical system 94 and the first image sensor 95a, and between the imaging optical system 94 and the second image sensor 95b. Therefore, the back focus of the imaging optical system 94 cannot be made smaller than the size of the dichroic prism 96. If the size of the dichroic prism 96 is limited, the width and angle of view of the light beam passing therethrough, for example, are also limited. This results in limitations on the brightness of an image obtained and the imaging range. Since the imaging optical system 94 of a general camera standard does not have a sufficient back focus value, the dichroic prism 96 cannot be disposed in a case of using such an imaging optical system 94.

It is stipulated in the general camera standard that the lens unit and the camera are joined so that the optical axis of the imaging optical system 94 is perpendicular to the imaging surface of the image sensor 95. However, in the imaging device 90B, the optical axis of the imaging optical system 94 is inclined relative to the imaging surface of the first image sensor 95a. The same holds true for the optical axis of the imaging optical system 94 and the imaging surface of the second image sensor 95b. Due to such an arrangement relationship, the lens unit and camera of the general camera standard cannot be easily combined.

Furthermore, in the imaging device 90B, the dichroic prism 96 is arranged on the side of the first image sensor 95a and the second image sensor 95b with the imaging optical system 94 as the reference. The convergent light beam converging from the imaging optical system 94 toward the first image sensor 95a passes through the dichroic prism 96. The same holds true for the convergent light beam converging from the imaging optical system 94 toward the second image sensor 95b.

Since the dichroic prism 96 is made of a dielectric material, a refraction phenomenon occurs when the convergent light beam enters and leaves the dichroic prism 96. When the convergent light beam passes through the dichroic prism 96, spherical aberration occurs due to this refraction phenomenon. Since the refractive index of the dielectric material is wavelength-dependent, chromatic aberration also occurs.

Both spherical aberration and chromatic aberration increase as the angular spreading of the convergent light beam increases. In the imaging device 90B, in order to suppress these aberrations, the distance between the imaging optical system 94 and the first image sensor 95a and the distance between the imaging optical system 94 and the second image sensor 95b need to be increased. This makes it difficult to downsize the imaging device 90B.

The inventor of the present disclosure has found the above problems and has come up with imaging devices according to embodiments of the present disclosure to solve these problems. The imaging devices according to Embodiments 1 and 2 will be described below. The imaging device according to Embodiment 1 obtains two images of a subject in different wavelength ranges, as an example of two images having different optical characteristics. The imaging device according to Embodiment 2 obtains two images of a subject having different polarization states, as another example of two images having different optical characteristics.

### Embodiment 1

### Imaging Device

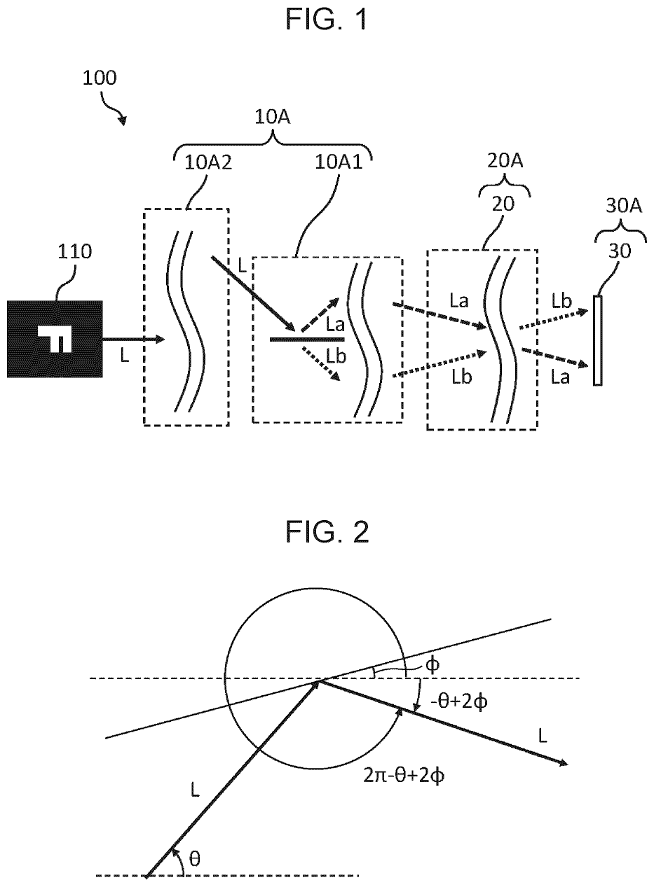

With reference to FIG. 1, a configuration example of an imaging device according to Embodiment 1 of the present disclosure will be described below. FIG. 1 is a diagram schematically illustrating a configuration of an imaging device according to exemplary Embodiment 1 of the present disclosure. FIG. 1 also illustrates a subject 110. An imaging device 100 illustrated in FIG. 1 obtains two images of the subject 110 in different wavelength ranges. As illustrated in FIG. 1, the imaging device 100 includes an optical component 10A including a first subcomponent 10A1 and a second subcomponent 10A2, a lens unit 20A including an imaging optical system 20, and a camera 30A including an image sensor 30. Note, however, that the second subcomponent 10A2 is not necessarily an essential component of the optical component 10A. The imaging device 100 has a simple configuration in which the optical component 10A is added to the lens unit 20A and the camera 30A.

The first subcomponent 10A1 includes at least one optical element. The same holds true for the second subcomponent 10A2. In FIG. 1, the first subcomponent 10A1, the second subcomponent 10A2, and the imaging optical system 20 are illustrated in an abstract form.

The first subcomponent 10A1 has a dichroic surface that separates a light beam L from the subject 110 into a first light beam La having a wavelength included in a first wavelength range and a second light beam Lb having a wavelength included in a second wavelength range. The dichroic surface is disposed in a plane including an optical axis of the imaging optical system 20. The solid arrows in FIG. 1 represent the light beam L from the subject 110. The dashed arrows in FIG. 1 represent the first light beam La having the wavelength included in the first wavelength range. The dotted arrows in FIG. 1 represent the second light beam Lb having the wavelength included in the second wavelength range. The first and second wavelength ranges are different from each other.

The first subcomponent 10A1 further converts the directions of the first light beam La having the wavelength included in the first wavelength range and the second light beam Lb having the wavelength included in the second wavelength range, which distance themselves from each other as they move away from the dichroic surface, and emits those light beams toward the imaging optical system 20. The angle between the two light beams after conversion is made smaller than the angle between the two light beams before conversion, thus making it easier to make both light beams incident on the imaging optical system 20.

The imaging optical system 20 forms images of the first light beam La and the second light beam Lb, which are incident on the imaging optical system 20, on the image sensor 30, thus forming an image of the first wavelength range and an image of the second wavelength range at different positions on the image sensor 30. The image of the first wavelength range and the image of the second wavelength range formed on the image sensor 30 are in mirror symmetric relationship.

However, since the dichroic surface of the first subcomponent 10A1 is disposed on the plane including the optical axis of the imaging optical system 20, the direction of the light beam L incident on the dichroic surface cannot be parallel to the optical axis of the imaging optical system 20.

Therefore, the second subcomponent 10A2 may be arranged between the subject 110 and the first subcomponent 10A1. The second subcomponent 10A2 emits the light beam L from the subject 110 in a direction different from the incident direction, and makes the light beam L from the subject 110 incident on the dichroic surface. This makes it possible to make the light beam L from the subject 110 incident on the dichroic surface even if the subject 110 is located almost on an extension line of the optical axis of the imaging optical system 20. As a result, the direction in which the subject 110 is located can almost coincide with the optical axis direction of the imaging optical system 20, making it possible to perform imaging in a natural direction.

Specific configurations of the first subcomponent 10A1 and the second subcomponent 10A2 will be described later.

In this specification, a light beam having a wavelength included in the first wavelength range is also referred to as a “first light beam”, and a light beam having a wavelength included in the second wavelength range is also referred to as a “second light beam”. The image of the first wavelength range is also referred to as a “first image”, and the image of the second wavelength range is also referred to as a “second image”.

A change in a traveling direction of a light beam in an actual optical element is caused by refraction or reflection. The change in the traveling direction of the light beam due to refraction can depend on, for example, a refractive index of the optical element, an incident angle at which the light beam enters the incident surface of the optical element, and an exit angle at which the light beam exits from the exit surface of the optical element. The closer the incident angle and the exit angle are to a perpendicular angle with respect to the incident surface and the exit surface, respectively, the smaller the change in the traveling direction of the light due to refraction. If the incident angle and the exit angle are completely perpendicular, the change in the traveling direction of the light due to refraction is zero. For simplicity, the change in the traveling direction of the light beam due to refraction is ignored in the following description.

Light reflection is caused by metal, a dielectric multilayer film, or total reflection. The reflection caused by the dielectric multilayer film other than metal and total reflection has conditions for the angle range or wavelength range for the reflection to occur. These conditions are well known and can be easily understood by those skilled in the art, and therefore description thereof is omitted. When it is stated in this specification that reflection occurs, it is assumed that the conditions are met.

A method of defining the traveling direction of the light beam L, the first light beam La, and the second light beam Lb is as follows. Here, the traveling direction of the light beam L is taken as an example. FIG. 2 is a diagram schematically illustrating how the light beam L is reflected by a mirror surface. As illustrated in FIG. 2, when the light beam L is reflected by the mirror surface, the traveling direction of the light beam L changes to a direction that is mirror-image inverted with respect to a plane perpendicular to the mirror surface. The traveling direction of the light beam L is defined by an angle that is positive in the counterclockwise direction from a reference direction, where the reference direction is the direction from left to right on the page space. The curved arrows in FIG. 2 indicate clockwise or counterclockwise. When the light beam L traveling in a direction θ is reflected by a mirror surface that forms an angle φ with the reference direction, the traveling direction of the light beam L after reflection changes to −θ+2φ or 2π−θ+2φ, where is the circular constant, and the unit of the angle including π is the radian.

Hereinafter, changes in the traveling directions of the light beam L, the first light beam La, and the second light beam Lb will be described based on the above general rule. The angles representing the traveling directions of the light beam L, the first light beam La, and the second light beam Lb can have both positive and negative values. As illustrated in FIG. 2, two angles with a difference of 2nπ (n is an integer) represent the same traveling direction. The other angles basically each have a positive value. Such an angle may be the angle between two faces of a prism, for example.

### Specific Configuration Example of Imaging Device

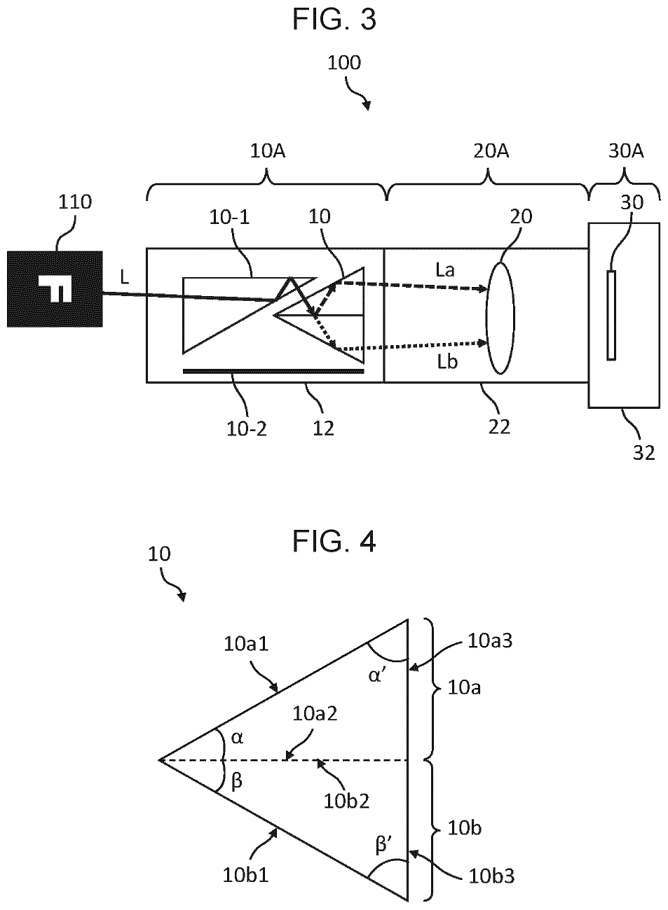

A specific configuration example of the imaging device according to Embodiment 1 of the present disclosure will be described below with reference to FIG. 3. FIG. 3 is a diagram schematically illustrating the specific configuration of the imaging device according to exemplary Embodiment 1 of the present disclosure. The subject 110 is also illustrated in FIG. 3. The imaging device 100 illustrated in FIG. 3 obtains two images of the subject 110 in different wavelength ranges. As illustrated in FIG. 3, the imaging device 100 includes the optical component 10A, the lens unit 20A, and the camera 30A.

The optical component 10A includes a dichroic prism 10, an optical path adjusting element 10-1, a light shielding body 10-2, and a housing 12 that houses them. Note, however, that the optical path adjusting element 10-1, the light shielding body 10-2, and the housing 12 are not essential components. The dichroic prism 10 corresponds to the first subcomponent 10A1 illustrated in FIG. 1. The optical path adjusting element 10-1 corresponds to the second subcomponent 10A2 illustrated in FIG. 1.

In this specification, the dichroic prism 10 is also referred to as a “first optical element” and the optical path adjusting element 10-1 is also referred to as a “second optical element”.

The lens unit 20A includes the imaging optical system 20 and a lens housing 22 that houses the imaging optical system 20. The camera 30A includes the image sensor 30 and a camera housing 32 that houses the image sensor 30. Note, however, that the lens housing 22 and the camera housing 32 are not essential components. The lens unit 20A and the camera 30A may be, for example, a general commercially available lens unit and a general commercially available camera.

The imaging device 100 includes at least the dichroic prism 10, the imaging optical system 20, and the image sensor 30. The imaging device 100 may further include other components as necessary.

Note that the imaging optical system 20 may include a plurality of lenses, but may be illustrated as a single lens or two lenses, for simplicity. The shape of the illustrated lens is also unrelated to the shape of an actual lens. The direction and spread of the light beam may also be illustrated in an exaggerated manner.

As will be described in detail later, in the imaging device 100, the dichroic prism 10 separates the light beam L from the subject 110 into the first light beam La and the second light beam Lb, and emits the first light beam La and the second light beam Lb symmetrically with respect to a certain plane, as illustrated in FIG. 3. The plane includes the optical axis of the imaging optical system 20. The imaging optical system 20 forms images of the first light beam La and the second light beam Lb, which are incident on the imaging optical system 20 at different angles, on the imaging surface of the image sensor 30, thus forming a first image and a second image at different positions on the imaging surface.

The first and second images on the imaging surface are not in a translation relationship but in a mirror-symmetric relationship. This makes it possible to reduce the influence of aberration. Therefore, the first and second images are suitable for evaluation of the subject 110 in two-color thermography, fluorescence imaging, and the like. Furthermore, the above configuration having the optical component 10A added to the lens unit 20A and the camera 30A is a simple configuration.

As described above, the imaging device 100 according to Embodiment 1 can obtain two images in different wavelength ranges suitable for evaluating the subject 110 with a simple configuration. The imaging device 100 according to Embodiment 1 may also have the optical path adjusting element 10-1 disposed between the subject 110 and the dichroic prism 10. The optical path adjusting element 10-1 changes the direction of the light beam L from the subject 110 and makes it incident on the dichroic prism 10. This makes it possible to perform imaging of the subject 110 in a natural orientation in which the subject 110 is located in the optical axis direction of the imaging optical system 20 and faces the image sensor 30.

The components of the imaging device 100 will be described in detail below.

### Components of Imaging Device 100

### Dichroic Prism 10

FIG. 4 is a diagram schematically illustrating an example of the dichroic prism 10. As illustrated in FIG. 4, the dichroic prism 10 includes a first Littrow prism 10a and a second Littrow prism 10b. The first Littrow prism 10a has a face 10a1, a face 10a2, and a face 10a3. The second Littrow prism 10b has a face 10b1, a face 10b2, and a face 10b3.

The first Littrow prism 10a has a triangular prism shape, and has two bottom faces located on opposite sides, in addition to the faces 10a1 to 10a3. Note, however, that the two bottom faces do not contribute to operations, and thus description thereof will be omitted. The two bottom faces of the first Littrow prism 10a may be painted in black to prevent reflection. The same applies to two bottom faces of the second Littrow prism 10b. It is assumed that the first Littrow prism 10a and the second Littrow prism 10b each have a sufficient thickness to form an image of the light beam L from the subject on the image sensor 30.

In the first Littrow prism 10a, the face 10a1 and the face 10a2 form an angle α, and the face 10a1 and the face 10a3 form an angle α′. In the second Littrow prism 10b, the face 10b1 and the face 10b2 form an angle β, and the face 10b1 and the face 10b3 form an angle β′. These angles have positive values. Since the Littrow prisms are used, the angles α and β are each 30°, and the angles α′ and β′ are each 60°. However, the Littrow prisms do not necessarily have to be used, and the values of three interior angles of a prism are arbitrary. The angles α and β may be equal to each other or different from each other. The angles α′ and β′ may be equal to each other or different from each other. In the following description, for ease of explanation, it is assumed that the angles α and β are equal to each other, and the angles α′ and β′ are equal to each other.

The faces 10a2 and 10b2 are bonded together with an adhesive layer. The adhesive layer is translucent in the second wavelength range. The faces 10a2 and 10b2 may be parallel to each other, for example. As a result of bonding the faces 10a2 and 10b2 together, the first Littrow prism 10a and the second Littrow prism 10b are arranged so as to be mirror-image symmetrical to each other.

One or both of the faces 10a2 and 10b2 may be dichroic surfaces that reflect the first light beam La and transmit the second light beam Lb of the light beam L incident from a direction in a specific range, for example. The dichroic surface has the above function in a state where the first Littrow prism 10a and the second Littrow prism 10b are bonded together. Here, the direction in the specific range refers to a direction in a range where the light beam L from the subject 110 is incident. The dichroic prism 10 thus has at least one dichroic surface as described above.

It is assumed in the following description that the face 10a2 is the dichroic surface, the direction in the specific range is a direction close to the normal to the face 10a1, and the faces 10a2 and 10b2 are parallel to each other. The face 10b1 is a reflecting surface that reflects the second light beam Lb. The face 10b1 may have, for example, a metal film of gold, silver, aluminum or the like, or a dielectric multilayer film. The metal film and the dielectric multilayer film have a high reflectivity of 60% or more or 80% or more for the second light beam Lb. The reflection of the second light beam Lb by the face 10b1 may be caused by the metal film or the dielectric multilayer film. Alternatively, the reflection may be caused by total reflection due to a difference between the refractive index of the material constituting the second Littrow prism 10b and the external refractive index.

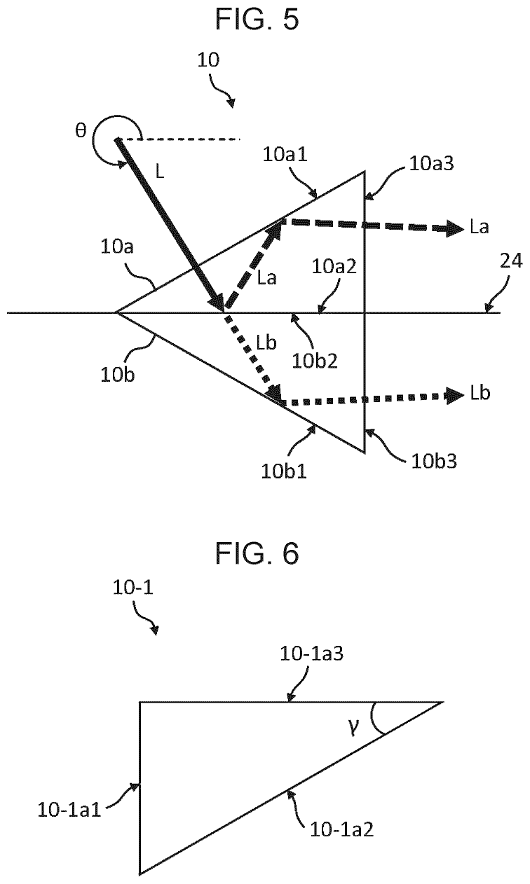

The paths of the light beam L, the first light beam La, and the second light beam Lb within the dichroic prism 10 will now be described. FIG. 5 is a diagram schematically illustrating an example of the paths of the light beams L, La, and Lb within the dichroic prism 10. The light beam L, the first light beam La, and the second light beam Lb illustrated in FIG. 5 represent only the optical path of the principal ray. An actual dichroic prism 10 transmit the light beam L, the first light beam La, and the second light beam Lb, each having a width and an angle range. In this case, the symmetry and positional relationship required for the faces 10a1 to 10a3 and the faces 10b1 to 10b3 are also as described above. The size of the dichroic prism 10 is appropriately designed according to the widths of the light beam L, the first light beam La, and the second light beam Lb, and the specifications of the imaging optical system 20.

In the first Littrow prism 10a, the light beam L incident on the face 10a1 from outside reaches the face 10a2, as illustrated in FIG. 5. The component of the light beam L in the first wavelength range is reflected by the face 10a2, which is the dichroic surface, to become the first light beam La. The first light beam La is reflected by the face 10a1 and emitted to the outside from the face 10a3. The reflection of the first light beam La by the face 10a1 is total reflection, and the incidence angle of the first light beam La on the face 10a1 is greater than or equal to a critical angle.

The component of the light beam L in the second wavelength range passes through the face 10a2, which is the dichroic surface, and the adhesive layer, to become the second light beam Lb in the second Littrow prism 10b. The second light beam Lb is reflected by the face 10b1 and emitted to the outside from the face 10b3. The reflection of the second light beam Lb by the face 10b1 is total reflection or reflection by the metal film or dielectric multilayer film.

In the second Littrow prism 10b, when the light beam L is incident on the face 10b1 from outside, the first light beam La can be emitted from the face 10b3 and the second light beam Lb can be emitted from the face 10a3, symmetrically to the above light beam L. However, the light shielding body 10-2 illustrated in FIG. 3 prevents the light beam L from entering the face 10b1 from outside. This causes the first light beam La to be emitted from the face 10a3 of the dichroic prism 10, and the second light beam Lb to be emitted from the face 10b3 of the dichroic prism 10.

As described above, the dichroic prism 10 separates the light beam L into the first light beam La and the second light beam Lb by the face 10a2. The dichroic prism 10 further converts the directions of the first light beam La and the second light beam Lb by the faces 10a1 and 10b1 before emitting them.

In the dichroic prism 10, the angles α and β are equal to each other, and the angles α′ and β′ are equal to each other. Therefore, the optical path of the first light beam La and the optical path of the second light beam Lb are substantially symmetrical with respect to the face 10a2.

Here, the expression “substantially symmetrical with respect to the face 10a2” means that, in the following cases, differences due to the wavelength dependency of the refraction phenomenon of the light beam L, the first light beam La, and the second light beam Lb can be ignored. These cases include cases where the second light beam Lb of the light beam L enters the second Littrow prism 10b from the first Littrow prism 10a, where the first light beam La is emitted from the first Littrow prism 10a, and where the second light beam Lb is emitted from the second Littrow prism 10b. The refraction phenomenon of the light beam L, the first light beam La, and the second light beam Lb depends on the incidence angle of the light beam L on the face 10a2, the incidence angle of the first light beam La on the face 10a3, and the incidence angle of the second light beam Lb on the face 10b3. The closer these incidence angles are to a right angle, the smaller the refraction phenomenon.

The first Littrow prism 10a and the second Littrow prism 10b may be designed so that the exit angle of the first light beam La from the face 10a3 and the exit angle of the second light beam Lb from the face 10b3 are close to the right angle. Alternatively, a glass material with a small difference in refractive index between the first and second wavelength ranges may be selected for the first Littrow prism 10a and the second Littrow prism 10b. Selecting such a glass material makes it possible to reduce the possibility that the first light beam La and the second light beam Lb deviate from the symmetrical relationship.

The traveling directions of the first light beam La and the second light beam Lb are as follows. With the face 10a2 as the reference of angle, the traveling direction of the light beam L incident on the face 10a1 is θ. In that case, the traveling direction of the first light beam La reflected by the face 10a2 is −θ, and the traveling direction of the first light beam La reflected by the face 10a1 is θ+2α. The traveling direction of the second light beam Lb reflected by the face 10b1 is −θ−2α.

Particularly, when the incident direction of the light beam Lis θ=−2α, the traveling direction of the first light beam La is θ+2α=0, and the traveling direction of the second light beam Lb is −θ−2α=0. Therefore, the first light beam La and the second light beam Lb emitted from the dichroic prism 10 are both parallel to the face 10a2.

When the incident direction of the light beam L is θ>−2α, the traveling direction of the first light beam La is θ+2α>0, and the traveling direction of the second light beam Lb is −θ−2α<0. Therefore, the first light beam La and the second light beam Lb move away from each other as they travel.

When the incident direction of the light beam L is θ<−2α, the traveling direction of the first light beam La is θ+2α<0, and the traveling direction of the second light beam Lb is −θ−2α>0. Therefore, the first light beam La and the second light beam Lb move closer to each other as they travel.

As illustrated in FIG. 3, the first light beam La and the second light beam Lb are both incident on the imaging optical system 20. The dichroic prism 10 and the imaging optical system 20 are arranged so as to satisfy the following condition. The condition is that the first light beam La and the second light beam Lb are incident on the imaging optical system 20 so as to be substantially symmetric with respect to one plane 24 including the optical axis of the imaging optical system 20, as illustrated in FIG. 5. When the plane 24 includes the face 10a2, the condition is satisfied.

### Imaging Optical System 20

The imaging optical system 20 is substantially symmetric with respect to the plane 24, and has optically symmetric characteristics with respect to the optical axis. The first light beam La and the second light beam Lb are incident on the imaging optical system 20 at different angles so as to be symmetric with respect to the plane 24. The optical axis of the first light beam La and the optical axis of the second light beam Lb are not parallel to each other. The imaging optical system 20 has refractive power, and forms a first image by imaging the first light beam La, and forms a second image by imaging the second light beam Lb. Due to the symmetric incidence of the first light beam La and the second light beam Lb, the first and second images are in a mirror-symmetric relationship with each other, as described below.

Here, the expression “the imaging optical system 20 is substantially symmetric with respect to the plane 24” means that the elements of the imaging optical system 20 that significantly affect the symmetry of the shape and brightness of the first and second images are substantially symmetric with respect to the plane 24. Due to manufacturing tolerances, there may be fluctuations in shape, decentering, and minute positional displacement.

It goes without saying that the arrangement of elements that do not affect the symmetry of the first and second images, such as the housing 12, the lens housing 22, the camera housing 32, and electrical circuits, is irrelevant. Even if the aperture has a polygonal shape, for example, and the symmetry is not strictly satisfied, strict symmetry does not have to be satisfied if the effect on the symmetry of the shape and brightness of the images is substantially negligible.

The imaging optical system 20 is arranged so that the plane 24 includes a normal vector of the imaging surface of the image sensor 30. This includes not only a case where the optical axis of the imaging optical system 20 and the normal vector of the imaging surface of the image sensor 30 are parallel, but also a so-called tilt shooting case where the optical axis of the imaging optical system 20 and the normal vector of the imaging surface of the image sensor 30 are non-parallel. The symmetry of the first light beam La and the second light beam Lb is maintained even in the tilt shooting.

If the imaging range of the image sensor 30 has a symmetrical shape, the plane 24 may include an axis of symmetry or a point of symmetry of the imaging range. For example, if the imaging range has a rectangular shape, the plane 24 may be located so as to be parallel to the short side of the imaging range and pass through the midpoint of the long side.

The imaging optical system 20 may have, for example, substantially the same optical characteristics in the first and second wavelength ranges. The optical characteristics may be, for example, chromatic aberration of magnification, axial chromatic aberration, field curvature, and peripheral darkening.

When both the chromatic aberration of magnification and axial chromatic aberration are small in the first and second wavelength ranges, corresponding positions in the first and second images can be easily identified. In the case of using the imaging optical system 20 with known characteristics of chromatic aberration of magnification, corresponding positions in the first and second images may be identified based on known information.

When the field curvature is the same in the first and second wavelength ranges, corresponding positions in the first and second images can be easily identified. The same applies to peripheral darkening in the first and second wavelength ranges.

### Image Sensor 30

The image sensor 30 has the imaging surface. The first and second images are formed at different positions on the imaging surface. The first and second images are in a mirror-symmetric relationship. Even the single image sensor 30 can obtain the first and second images thus formed.

The image sensor 30 includes a plurality of photoelectric conversion elements arranged one-dimensionally or two-dimensionally. The plurality of photoelectric conversion elements form the imaging surface. The photoelectric conversion elements have sensitivity in each of the first and second wavelength ranges. The photoelectric conversion elements convert the light intensity at each point in the first and second images into an electric signal.

The photoelectric conversion elements may have the same sensitivity or different sensitivities in the first and second wavelength ranges. In the case of imaging thermal radiation in the first and second wavelength ranges, one of the intensities of thermal radiation in the first and second wavelength ranges emitted from the subject 110 is often relatively high and the other relatively low. In that case, the sensitivity in the wavelength range with the relatively high intensity may be set low, and the sensitivity in the wavelength range with the relatively low intensity may be set high. Such sensitivity adjustment allows the signal intensities of the both to become closer. This makes it possible to reduce the possibility of one becoming saturated due to excessive signal intensity, or the other becoming buried in noise due to insufficient signal intensity.

When the image sensor 30 includes a plurality of photoelectric conversion elements arranged one-dimensionally, the camera 30A may further include a scanning device for obtaining a two-dimensional image. The camera 30A may further include a filter array having a specific optical function, as described later.

The camera 30A includes a control circuit for controlling the operation of the image sensor 30. The control circuit obtains the electric signal outputted from each photoelectric conversion element and outputs image data.

In the imaging device 100, the lens unit 20A and the camera 30A do not require any special elements or configurations other than those used for general imaging. This makes it possible to use a general lens unit and a general camera in any combination in the imaging device 100. Since the lens unit 20A and the camera 30A are detachable, such use in any combination is possible.

As the lens unit 20A and the camera 30A, a lens unit and a camera conforming to standards such as C mount and F mount can be used. In this case, one or both of the lens unit 20A and the camera 30A can be replaced within a range conforming to the standards. Such replacement is effective when changing the focal length of the lens to change the imaging range according to the size of the subject 110, or when changing to a high frame rate camera for high speed shooting.

### Optical Path Adjusting Element 10-1

The optical path adjusting element 10-1 is provided as an auxiliary element if necessary. For example, as illustrated in FIG. 3, the optical path adjusting element 10-1 changes the direction of the light beam L from the subject 110 and makes the light beam L enter the dichroic prism 10. The optical path adjusting element may include a reflecting prism such as a Littrow prism and a half pentaprism, for example, and a mirror.

When the optical path adjusting element 10-1 is not provided, the subject 110 is placed diagonally above the imaging optical system 20, which is a direction non-parallel to the optical axis of the imaging optical system 20, in the example illustrated in FIG. 3. When combining a general lens unit and a general camera, the subject 110 is almost always located in the direction of the optical axis of the imaging optical system 20. Therefore, it is counterintuitive to place the subject 110 diagonally above the imaging optical system 20. This problem can be solved by arranging the optical path adjusting element 10-1 between the subject 110 and the dichroic prism 10.

The optical path adjusting element 10-1 reflects or totally reflects the light beam L incident in a direction roughly parallel to the optical axis of the imaging optical system 20 to emit the light beam L in a direction different from its original direction. By appropriately designing the optical path adjusting element 10-1, the first light beam La and the second light beam Lb separated from the light beam L by the dichroic prism 10 are emitted from the dichroic prism 10 at appropriate angles. As a result, the imaging optical system 20 can form images of the first light beam La and the second light beam Lb, thus forming the first and second images at desired positions on the image sensor 30. The optical path adjusting element 10-1 can be designed, for example, by adjusting the angle of the face at which reflection or total reflection occurs.

FIG. 6 is a diagram schematically illustrating an example of the optical path adjusting element 10-1. In the example illustrated in FIG. 6, the optical path adjusting element 10-1 is a Littrow prism having a triangular prism shape. The optical path adjusting element 10-1 has a face 10-1a1, a face 10-1a2, and a face 10-1a3 as side surfaces. The optical path adjusting element 10-1 further has two bottom faces located on opposite sides, in addition to the faces 10-1a1 to 10-1a3. Note, however, that the two bottom faces do not contribute to the operation, and thus description thereof will be omitted. The face 10-1a2 and the face 10-1a3 form an angle γ. Since the Littrow prism is used, the angle γ is π/6. However, the Littrow prism does not necessarily have to be used, and the values of three interior angles of the optical path adjusting element 10-1 are arbitrary. The face 10-1a3 may have a metal film or a dielectric multilayer film to achieve a high light reflectivity.

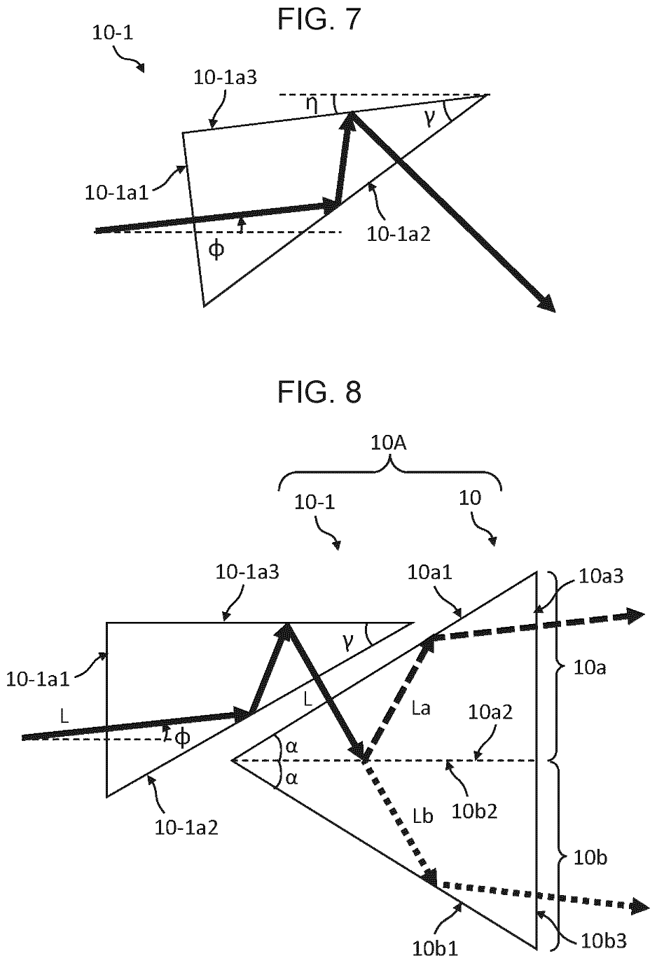

FIG. 7 is a diagram schematically illustrating an example of the path of the light beam L within the optical path adjusting element 10-1. As illustrated in FIG. 7, the face 10-1a3 is inclined at an angle n with respect to the face 10a2 illustrated in FIG. 5.

The light beam L in the traveling direction q is incident on the face 10-1a1 and then totally reflected by the face 10-1a2. Since the face 10-1a2 is inclined at an angle η+γ with respect to the face 10a2, the traveling direction of the light beam L totally reflected by the face 10-1a2 is −φ+2 (η+γ). The light beam L totally reflected by the face 10-1a2 is totally reflected by the face 10-1a3. The traveling direction of the light beam L totally reflected by the face 10-1a3 is −(−φ+2 (η+γ))+2η=φ−2γ. Specifically, the traveling direction of the light beam L totally reflected by the face 10-1a3 is rotated by an angle of −2γ from the traveling direction of the light beam L incident on the face 10-1a1, and is not dependent on the angle n. This is because the effect of the angle n is offset by the reflections on the face 10-1a2 and the face 10-1a3.

This phenomenon occurs in all prisms in which the light beam L, the first light beam La, and the second light beam Lb are reflected twice on the inner surface of the prism. Therefore, the following description has no particular mention of the arrangement angle of the prism, when the light beam L, the first light beam La, and the second light beam Lb are reflected twice on the inner surface of the prism.

The imaging device 100 may be provided with at least one of the following optical filter, optical system, or light shielding body, instead of or in addition to the optical path adjusting element 10-1. The optical filter blocks or attenuates a light beam in a wavelength range other than the first and second wavelength ranges. The optical system corrects spherical aberration caused by the prism. The light shielding body limits the imaging range of the subject 110.

### Light Shielding Body 10-2

The light shielding body 10-2 is arranged as an auxiliary component if necessary. The light shielding body 10-2 blocks unintended light beams from entering the dichroic prism 10. This makes it possible to reduce stray light. As illustrated in FIG. 3, the light shielding body 10-2 is arranged so that the light beam L enters a specific face of the first Littrow prism 10a of the dichroic prism 10 illustrated in FIG. 5 from outside, and does not enter any other face. The specific face is the face 10a1 illustrated in FIG. 5. The light shielding body 10-2 blocks the light beam L from entering the face 10b1 of the second Littrow prism 10b of the dichroic prism 10 illustrated in FIG. 5 from outside. The light shielding body 10-2 further blocks the light beam L from entering the two bottom faces of the first Littrow prism 10a and the two bottom faces of the second Littrow prism 10b.

The light shielding body 10-2 may be, for example, a light shielding object arranged at a distance from the dichroic prism 10 as illustrated in FIG. 3. Alternatively, the light shielding body 10-2 may be, for example, a light shielding layer formed on a face of the dichroic prism 10 on which no light beam is to be made incident. The metal film or dielectric multilayer film on the face 10b1 that reflects the second light beam Lb may also be configured to function as the light shielding body 10-2.

### Paths of Light Beam L, First Light Beam La, and Second Light Beam Lb in Optical Component 10A

FIG. 8 is a diagram schematically illustrating the paths of the light beam L, the first light beam La, and the second light beam Lb in the optical component 10A. However, the light shielding body 10-2 and the housing 12 illustrated in FIG. 3 are omitted in FIG. 8. The light beam L incident on the optical path adjusting element 10-1 in the direction q is reflected on the inner surface and then finally emitted from the optical path adjusting element 10-1 in the direction φ−2γ.

The light beam L is incident on the face 10a1 of the dichroic prism 10 and separated into the first light beam La and the second light beam Lb by the face 10a2. Finally, the first light beam La exits the dichroic prism 10 in the direction φ−2γ+2α, and the second light beam Lb exits the dichroic prism 10 in the direction −φ+2γ−2α. Specifically, the optical component 10A emits the incident light beam L as the first light beam La and the second light beam Lb, which are in a mirror-image symmetry with respect to the face 10a2.

Particularly, when φ=2γ−2α is satisfied, the traveling directions of the first light beam La and the second light beam Lb emitted from the dichroic prism 10 are both zero. If the angle γ substantially matches the angle α, the direction φ is substantially zero. Therefore, when the traveling direction of the light beam L incident on the optical path adjusting element 10-1 is substantially parallel to the optical axis of the imaging optical system 20, the traveling directions of the first light beam La and the second light beam Lb emitted from the dichroic prism 10 are substantially parallel to the optical axis of the imaging optical system 20.

In this specification, the expression “the traveling directions of the light beam L, the first light beam La, and the second light beam Lb are substantially parallel to the optical axis direction of the imaging optical system 20” means not only the case where the traveling directions of the light beam L, the first light beam La, and the second light beam Lb are strictly parallel to the optical axis direction of the imaging optical system 20, but also the case where the angle between the traveling directions of the light beam L, the first light beam La, and the second light beam Lb and the optical axis direction of the imaging optical system 20 is smaller than or equal to π/36.

### General Characteristics of Imaging Optical System 20

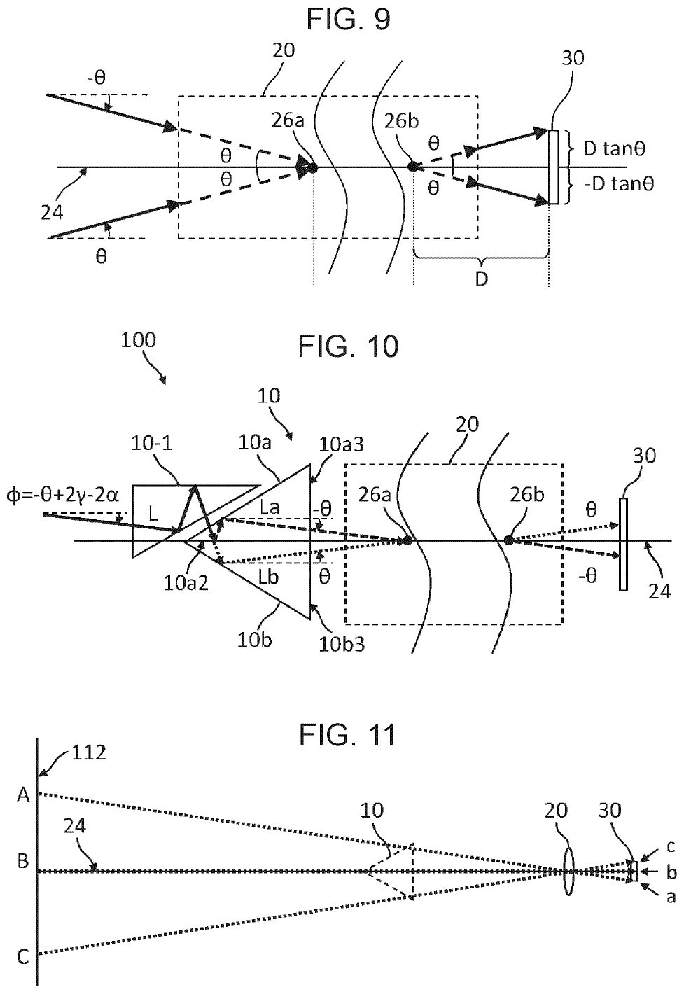

FIG. 9 is a diagram for explaining general characteristics of the imaging optical system 20. The imaging optical system 20 has a front principal point 26a and a rear principal point 26b on its optical axis.

Among light rays incident in a direction −θ on the optical axis of the imaging optical system 20, a light ray passing through the front principal point 26a is emitted in the direction −θ from the rear principal point 26b. Similarly, among light rays incident in a direction θ on the optical axis of the imaging optical system 20, a light ray passing through the front principal point 26a is emitted in the direction θ from the rear principal point 26b. Here, the light ray passing through the front principal point 26a is referred to as a principal ray.

The subject 110 that is imaged at a position D tan θ on the image sensor 30 with respect to the plane 24 is located in a direction θ+π with respect to the front principal point 26a, where D is the distance from the rear principal point 26b to the image sensor 30. Similarly, the subject 110 that is imaged at a position −D tan θ on the image sensor 30 with respect to the plane 24 is located in a direction −θ−π with respect to the front principal point 26a. From the above principal ray, the relationship between the direction of the subject 110 and the imaging position on the image sensor 30 can be known.

Paths of Light beam L, First Light Beam La, and Second Light Beam Lb in Imaging Device 100

FIG. 10 is a diagram schematically illustrating an example of the paths of the light beam L, the first light beam La, and the second light beam Lb in the imaging device 100. Note, however, that the light shielding body 10-2, the housing 12, the lens housing 22, and the camera housing 32 illustrated in FIG. 3 are omitted in FIG. 10. From the relationship between the incident direction of the light beam L and the emitting directions of the first light beam La and the second light beam Lb, the relationship between the direction in which the subject 110 is located and the imaging position can be determined.

As illustrated in FIG. 10, when the light beam L is incident on the optical path adjusting element 10-1 in the direction φ=−θ+2γ−2α, the first light beam La is incident in the direction −θ on the imaging optical system 20 and passes through the front principal point 26a, and the second light beam Lb is incident in the direction θ on the imaging optical system 20 and passes through the front principal point 26a. In this case, the first light beam La is imaged at a position of −D tan θ on the image sensor 30, and the second light beam Lb is imaged at a position of D tan θ on the image sensor 30. In other words, the first and second images on the image sensor 30 are in a mirror-image symmetry with respect to the plane including the face 10a2.

The first light beam La is emitted from the face 10a3 of the first Littrow prism 10a. Therefore, when the traveling direction −θ of the first light beam La is negative, the first light beam La may include a principal ray passing through the front principal point 26a. On the other hand, when the traveling direction −θ of the first light beam La is positive, the first light beam La does not include such a principal ray. With respect to the plane 24, the first image is formed on the second Littrow prism 10b side on the image sensor 30. The first image may be formed on the first Littrow prism 10a side on the image sensor 30, but is generally darkened due to the effect of vignetting.

The second light beam Lb is emitted from the face 10b3 of the second Littrow prism 10b. Therefore, when the traveling direction θ of the second light beam Lb is positive, the second light beam Lb may include a principal ray passing through the front principal point 26a. When the traveling direction θ of the second light beam Lb is negative, the second light beam Lb does not include such a principal ray. With respect to the plane 24, the second image is formed on the first Littrow prism 10a side on the image sensor 30. The second image may be formed on the second Littrow prism 10b side on the image sensor 30, but is generally darkened due to the effect of vignetting.

Therefore, the areas on the image sensor 30 where the first and second images are mainly formed are different.

When the direction θ is zero, the subject 110 located in the direction φ=2γ−2α is imaged to form first and second images at the position where the optical axis of the imaging optical system 20 intersects the image sensor 30. In other words, the subject 110 located in the direction φ=2γ−2α is imaged at the center of the image sensor 30.

Here, when α=γ, the direction in which the subject 110 is located is φ=0. Therefore, the light beam L from the subject 110 located in the optical axis direction of the imaging optical system 20 is imaged to form first and second images at the position where the optical axis of the imaging optical system 20 intersects the image sensor 30. The center of the area where the first and second images are formed is located in the direction of θ=0.

On the other hand, when α≠γ, the direction in which the subject 110 is located is φ≠0. Conversely, the light beam L incident in the direction φ≠0 becomes the first light beam La emitted in the direction −θ=−2γ+2α and the second light beam Lb emitted in the direction θ=2γ−2α. Such a configuration makes it possible to form images of the first light beam La and the second light beam Lb at different positions on the image sensor 30, when the subject 110 is located on the optical axis of the imaging optical system 20.

For example, the center of the range in which the first light beam La is imaged on the image sensor 30 is located at −D tan(2γ−2α), and the center of the range in which the second light beam Lb is imaged on the image sensor 30 is located at D tan(2γ−2α). Such selection of α and γ allows the center of the subject 110 corresponding to the respective centers of the first and second images to be positioned in the optical axis direction of the imaging optical system 20. This makes it possible to obtain the first and second images by orienting the imaging optical system 20 in a natural direction.

As illustrated in FIG. 10, the configuration in which the dichroic prism 10 and the optical path adjusting element 10-1 are combined is compact because the distance therebetween can be narrowed. The shape of the prism used in this configuration is also simple.

### Principle of Obtaining First and Second Images

Next, the principle of obtaining the first and second images by the imaging device 100 according to Embodiment 1 will be described. Hereinafter, description will be given of the positional relationship between respective parts in the subject 110 illustrated in FIG. 3 when imaging on the image sensor 30. Here, for ease of explanation, the optical path adjusting element 10-1 will not be taken into consideration.

FIG. 11 is a diagram schematically illustrating how a principal ray emitted from a certain point is imaged on the image sensor 30 when there is no dichroic prism 10. As illustrated in FIG. 11, when there is no dichroic prism 10, an image on a subject surface 112 is formed on the image sensor 30, where the subject surface 112 is a surface conjugate with the image sensor 30 by the imaging optical system 20.

The imaging range of the image sensor 30 as viewed from the imaging optical system 20 side is determined based on the focal length of the imaging optical system 20, the range where vignetting occurs, and the size of the image sensor 30. It is assumed in the following description that the imaging range has a rectangular shape and is symmetrical with respect to the plane 24.

If no mirror is disposed in the optical path, a position on the subject surface 112 that is conjugate with a point a on the image sensor 30 is defined as a point A, a position on the subject surface 112 that is conjugate with a point b on the image sensor 30 is defined as a point B, and a position on the subject surface 112 that is conjugate with a point c on the image sensor 30 is defined as a point C. The point b is located on the optical axis of the imaging optical system 20. The point a and the point c are located at the ends of the imaging range.

Specifically, an image of the subject 110 in the range on a line segment A-C on the subject surface 112 is obtained by the image sensor 30. A line segment a-b and a line segment b-c have the same length. A line segment A-B and a line segment B-C have the same length.

By disposing a mirror in the following position, the subject surface 112 is moved to the destination of reflection by the mirror. This position is on the opposite side of the image sensor 30 with respect to the imaging optical system 20. This position is closer to the imaging optical system 20 than the subject surface 112. This position is also on the optical path where imaging can be performed by the image sensor 30.

The subject 110 that is actually imaged is located on the moved subject surface. However, the subject 110 appears to be located on subject surface 112 if the mirror is ignored. In this specification, this position is referred to as an apparent subject position.

Hereinafter, description will be given of an example where the subject 110 has a planar shape, is located on a single plane perpendicular to the face 10a2 of the dichroic prism 10, and is orthogonal to the optical axis of the imaging optical system 20. As for the subject 110 that is not located on the single plane, the imaging result can be calculated using elementary geometry, and thus description thereof will be omitted.

### First Image

As for the first image, a positional relationship between an apparent subject and the actual subject 110 will be described. As will be described later, not all of the actual range of the subject 110 is imaged in the imaging range on the image sensor 30.

When the light beam L from the subject 110 is incident on the face 10a1 of the first Littrow prism 10a from outside as illustrated in FIG. 5, the face 10a1 and the face 10a2 function as mirror surfaces that reflect the first light beam La separated from the light beam L. On the other hand, when the light beam L from the subject 110 is incident on the face 10b1 of the second Littrow prism 10b from outside, the face 10b1 and the face 10b2 function as mirror surfaces that reflect the first light beam La separated from the light beam L. However, the light beam incident on the face 10b1 from outside is blocked by the light shielding body 10-2 illustrated in FIG. 3. Therefore, the latter first light beam La is not imaged on the image sensor 30. For this reason, the only first light beam La considered is the first light beam La separated from the light beam L incident on the face 10a1.

After being reflected by the face 10a2, the first light beam La is further reflected by the face 10a1 and then enters the imaging optical system 20. By tracing back the optical path of this first light beam La, the position of the actual subject 110 can be determined from the position of the apparent subject. Specifically, after determining the position of the intermediate apparent subject due to reflection on the face 10a1, the position of the actual subject 110 due to reflection on the face 10a2 is determined.

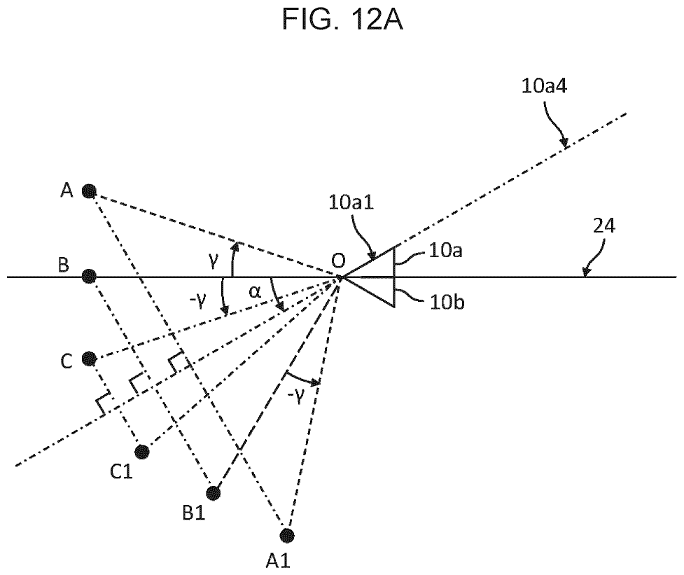

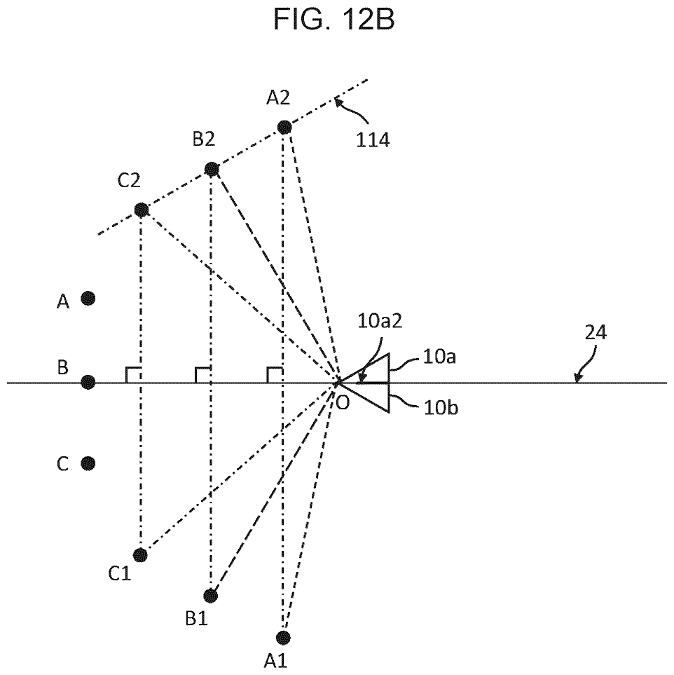

FIG. 12A is a diagram for explaining the position of the intermediate apparent subject due to reflection by the face 10a1 for the first image. As illustrated in FIG. 12A, a plane 10a4 is the plane including the face 10a1. A point O is the intersection point between the intersection line of the plane 10a4 and the plane 24 and the plane including the points A to C and the optical axis of the imaging optical system 20. φBOA is γ. γ as a negative value. Since a line segment B-A and a line segment B-C have the same length, ∠BOC is −γ. It is, however, assumed that the angle increases counterclockwise on the page space with the optical axis of the imaging optical system 20 as the zero reference.

When the optical path is viewed from the image sensor 30 side, the first reflecting surface is the face 10a1. When at least some of the light beams converging to the points a, b, or c illustrated in FIG. 11 are reflected by the face 10a1, the conjugate positions move to symmetric positions with respect to the face 10a1. As a result, as illustrated in FIG. 12A, the points A1, B1, and C1 become conjugate with the points a, b, and c, respectively.

∠BOA1 is 2α−γ, ∠BOB1 is 2α, and ∠BOC1 is 2α+γ. A line segment AO and a line segment A1O have the same length. Similarly, a line segment BO and a line segment B1O have the same length. A line segment CO and a line segment C1O have the same length. The positions of the points A1, B1, and C1 are intermediate apparent subject positions for the first light beam La.

FIG. 12B is a diagram for explaining the actual subject position due to reflection by the face 10a2 for the first image. As illustrated in FIG. 12B, due to reflection by the face 10a2, the points A1, B1, and C1 move to points A2, B2, and C2, respectively. The points A2, B2, and C2 are symmetrical to the points A1, B1, and C1, respectively, with respect to the plane 24. The subject 110 located at the points A2, B2, and C2 appears to be located at the apparent points A1, B1, and C1. The points A2, B2, and C2 exist on the actual subject surface 114.

A line segment A1O and a line segment A20 have the same length. Similarly, a line segment B1O and a line segment B20 have the same length. A line segment C1O and a line segment C20 have the same length. Therefore, the line segment AO and the line segment A20 have the same length. Similarly, the line segment BO and the line segment B20 have the same length. The line segment CO and the line segment C20 have the same length.

∠BOA2 is γ−2α, ∠BOB2 is −2α, and ∠BOC2 is −γ−2α. Specifically, the positions of the points A2, B2, and C2 are positions obtained by rotating the points A, B, and C by −2α around the point O, respectively.

In summary, for the first light beam La, the subject 110 that is actually located at the points A2, B2, and C2 appears to be located at the apparent points A, B, and C.

The subject 110 reflected by the reflecting surface becomes a so-called mirror-inverted image with each reflection. Therefore, an image that has undergone an odd number of reflections becomes a mirror-inverted image with respect to an image that has undergone no reflections. An image that has undergone an even number of reflections becomes a non-mirror-inverted image with respect to the image that has undergone no reflections.

Two mirror-image inversions are mathematically equivalent to one rotation. The first light beam La is reflected twice by the faces 10a1 and 10a2 of the dichroic prism 10. Therefore, the first image formed on the image sensor 30 is an image that is not mirror-inverted with respect to the image that has undergone no reflections. The position of the subject 110 that is actually imaged is at a position rotated by −2α from the apparent position of the subject, with the point O as the rotation axis.

### Second Image

As for the second image, a positional relationship between the apparent subject and the actual subject 110 will be described. As with the first light beam La, the light beam incident from the face 10b1 side is blocked. Therefore, as for the second light beam Lb, the only optical path considered is that of the second light beam Lb separated from the light beam L incident on the face 10a1 of the first Littrow prism 10a.

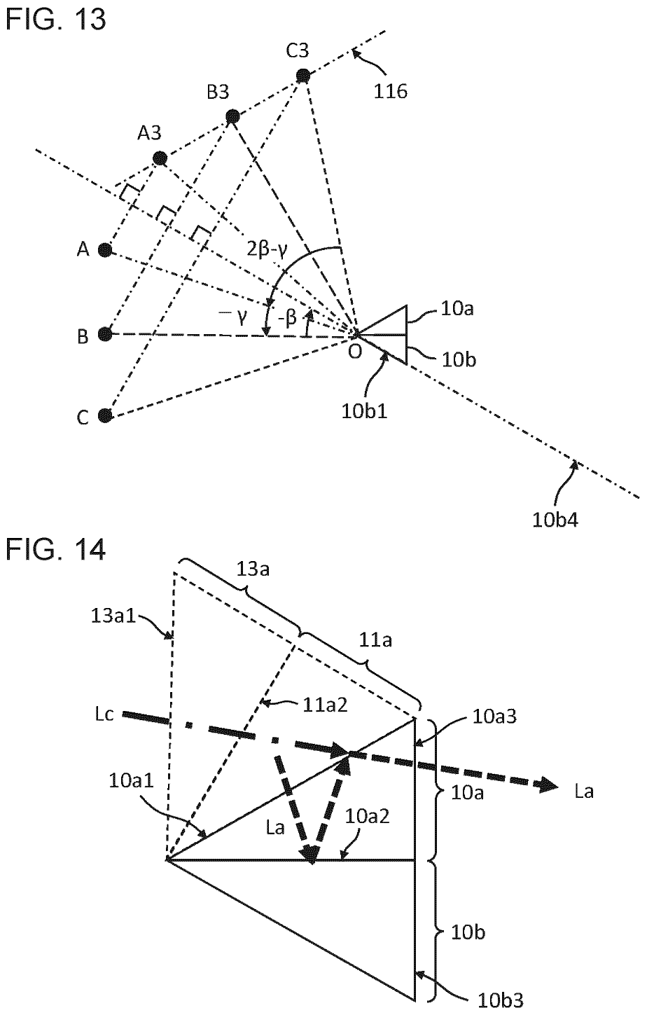

FIG. 13 is a diagram for explaining the position of the actual subject 110 due to reflection by the face 10b1 for the second image. When the optical path is viewed from the image sensor 30 side, the only reflecting surface is the face 10b1. As illustrated in FIG. 13, a plane 10b4 is the plane including the face 10b1. Points A3, B3, and C3 are symmetrical to points A, B, and C, respectively, with respect to the plane 10b4. The subject 110 located at the points A3, B3, and C3 appears to be located at the apparent points A, B, and C. The points A3, B3, and C3 exist on an actual subject surface 116.

∠BOA3 is −γ−2β, ∠BOB3 is −2β, and ∠BOC3 is γ−2β. When α=β∠BOA3 is −γ−2α, ∠BOB3 is −2α, and ∠BOC3 is γ−2α. A line segment AO and a line segment A3O have the same length. Similarly, a line segment BO and a line segment B3O have the same length. A line segment CO and a line segment C3O have the same length.

When the refractive power of the imaging optical system 20 is the same in the first and second wavelength ranges, the subject surface 116 illustrated in FIG. 13 is the same as the subject surface 114 illustrated in FIG. 12B. That is, the point A3 is the same as the point C2, the point B3 is the same as the point B2, and the point C3 is the same as the point A2.

The apparent subjects in the first and second wavelength ranges as seen from the actual subject 110 are mirror-image inverted from each other. That is, the first and second images formed on the image sensor 30 are mirror-image inverted from each other.

This is because the first light beam La is reflected an even number of times, twice, by the faces 10a1 and 10a2, while the second light beam Lb is reflected an odd number of times, once, by the face 10b1. Specifically, when one of the two numbers of reflections of the light beam is the even number of times and the other is the odd number of times, the two images are mirror-image inverted from each other.

In the above example, the optical path of the first light beam La and the optical path of the second light beam Lb have been described separately. The first light beam La and the second light beam Lb are actually imaged at the same time. As a result, the first and second images are obtained at the same time by the image sensor 30. This is because the first light beam La and the second light beam Lb have the same optical path length.

### Imaging Range of First Light Beam La and Second Light Beam Lb

The correspondence between the subject 110 and the first and second images on the image sensor 30 is as described above. The first and second images actually formed on the image sensor 30 differ depending on, for example, the relative positional relationship between the dichroic prism 10, the imaging optical system 20, the image sensor 30, the optical path adjusting element 10-1, and the light shielding body 10-2. The first and second images further differ depending on, for example, the size, focal length, and aperture setting value of these components.

A light ray emitted in a certain direction from a certain point in the subject 110 passes through the optical path adjusting element 10-1, the dichroic prism 10, and the imaging optical system 20, and reaches the imaging range on the image sensor 30. As a result, an image at the certain point in the subject 110 is obtained by the image sensor 30. If no light ray emitted in any direction from a certain part of the subject reaches the imaging range on the image sensor 30, no image of that part of the subject 110 is formed.

The range of the subject 110 that is actually imaged on the image sensor 30 will be described below. The imaging device 100 is configured so that only the light beam that has passed through both the dichroic prism 10 and the imaging optical system 20 reaches the imaging range on the image sensor 30. Such a configuration is possible by appropriately adjusting the size of the dichroic prism 10, the positional relationship between the dichroic prism 10 and the imaging optical system 20, the focal length of the imaging optical system 20, and the size and position of the light shielding body 10-2. Such a configuration will be assumed in the following description. Specifically, there is no light beam that enters the imaging optical system 20 without passing through the dichroic prism 10.

First, a range in which the first light beam La is actually imaged on the image sensor 30 will be described. FIG. 14 is a diagram schematically illustrating how the first light beam La travels through the first Littrow prism 10a. The light beam L from the subject 110 includes the first light beam La as a part thereof. As illustrated in FIG. 14, the first light beam La passes through the face 10a1 of the first Littrow prism 10a from outside and is reflected by the faces 10a2 and 10a1 in this order before passing through the face 10a3. Therefore, the light beam that is neither transmitted through nor reflected by the faces 10a1 to 10a3 is not imaged on the image sensor 30.

Tracing the optical path from the imaging optical system 20 side, the first light beam La passes through the face 10a3 and is reflected by the faces 10a1 and 10a2 in this order before passing through the face 10a1. Due to reflection by the face 10a1, the first Littrow prism 10a appears to be a prism 11a. The prism 11a is symmetrical to the first Littrow prism 10a with respect to a plane including the face 10a1. The face 10a2 appears to be a corresponding face 11a2 of the prism 11a.

Similarly, due to reflection by the face 10a2, the prism 11a appears to be a prism 13a. The prism 13a is symmetrical to the prism 11a with respect to a plane including the face 11a2. The face 10a1 as a passing surface appears to be a corresponding face 13al of the prism 13a.

Therefore, the first light beam La corresponds to an apparent light beam Lc passing from the apparent subject through the actual first Littrow prism 10a and through a thick plate combining the two apparent prisms 11a and 13a. The dashed-dotted arrow in FIG. 14 represents the light beam Lc. When there is the actual first light beam La passing through the first Littrow prism 10a, the apparent light beam Lc can be considered. On the other hand, when the apparent light beam Lc cannot be considered, there is no actual first light beam La.

The presence of a light beam passing through the face 13al of the apparent prism 13a and the face 10a3 of the first Littrow prism 10a is a prerequisite for imaging of the first light beam La.

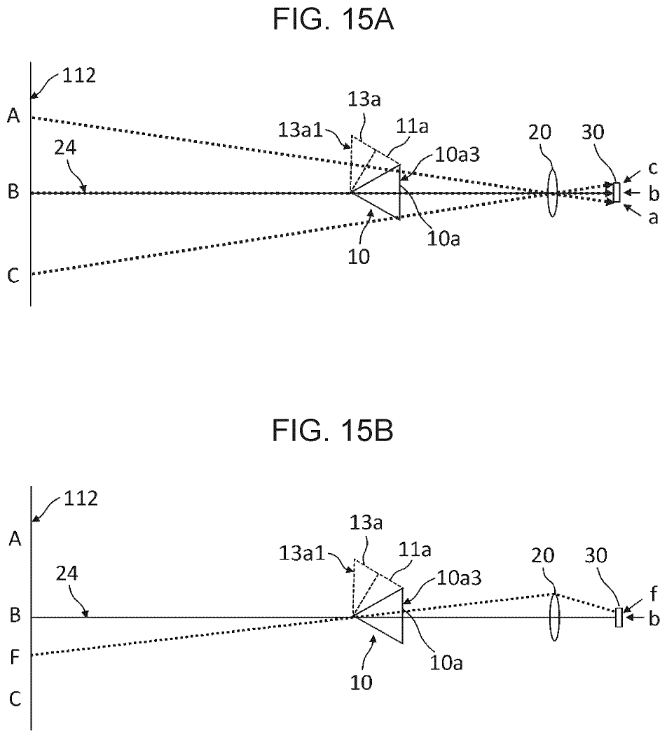

FIGS. 15A and 15B are diagrams each schematically illustrating how a light ray emitted from a certain point is imaged on the image sensor 30 when there are the apparent prisms 11a and 13a, in addition to the dichroic prism 10, for the first light beam La.

As illustrated in FIG. 15A, a line segment AB is located on the side of the face 13al and the face 10a3 through which the first light beam La is to pass, with respect to the plane 24. Therefore, a point on the line segment AB on the apparent subject can be imaged in the imaging range of the image sensor 30 by appropriately designing the size and position of the first Littrow prism 10a.

On the other hand, a line segment BC is on the opposite side to the face 13al and the face 10a3, with respect to the plane 24. Therefore, a principal ray emitted from a point on the line segment BC does not pass through the faces 13al and 10a3, regardless of the size and position of the first Littrow prism 10a. However, a light ray that has passed through an optical path other than the principal ray may be imaged in the imaging range of the image sensor 30 even if the light ray is emitted from the point on the line segment BC.

As illustrated in FIG. 15B, a point F is the apparent position that is the boundary of the range where the light ray that has passed through the faces 13al and 10a3 is imaged in the imaging range on the image sensor 30 by the imaging optical system 20. A point f is the position where the light ray that appears to be emitted from the point Fis imaged on the image sensor 30.

A point on a line segment B-F on the apparent subject is imaged on a line segment b-f in the imaging range of the image sensor 30 by the imaging optical system 20. A point on a line segment F-C on the apparent subject is not imaged in the imaging range on the image sensor 30, because a principal ray emitted from that point does not pass through the imaging optical system 20.

If no dichroic prism 10 is disposed, the range of a line segment A-C on the apparent subject is imaged in the imaging range of the image sensor 30. On the other hand, if the dichroic prism 10 is actually disposed, only the range of a line segment A-B and the line segment B-F on the apparent subject is imaged in the imaging range of the image sensor 30 for the first light beam La. The range of the line segment F-C on the apparent subject is not imaged because no light ray reaches the image sensor 30.

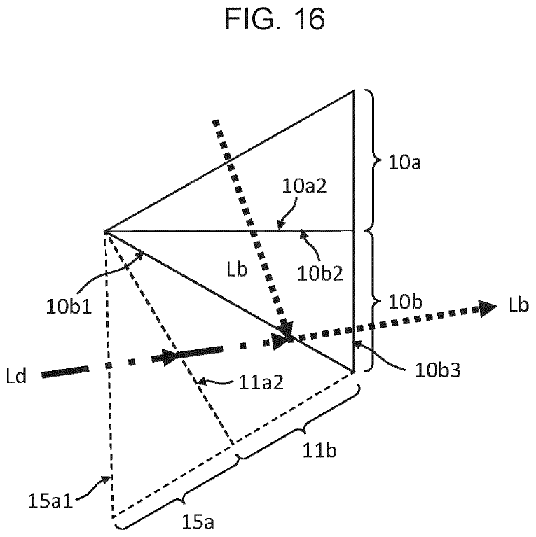

Next, a range in which the second light beam Lb is actually imaged on the image sensor 30 will be described. FIG. 16 is a diagram schematically illustrating how the second light beam Lb travels through the second Littrow prism 10b. The light beam L from the subject 110 includes the second light beam Lb as a part thereof. As illustrated in FIG. 16, the second light beam Lb passes through the faces 10a1, 10a2, and 10b2 of the first Littrow prism 10a from outside and is reflected by the face 10b1 of the second Littrow prism 10b before passing through the face 10b3. Therefore, the light beam that is neither transmitted through nor reflected by the faces 10a1, 10a2, and 10b1 to 10b3 is not imaged on the image sensor 30.

Tracing the optical path from the imaging optical system 20 side, the second light beam Lb passes through the face 10b3 and is reflected by the face 10b1 before passing through the faces 10b2, 10a2, and 10a1 in this order.

Due to reflection by the face 10b1, the second Littrow prism 10b appears to be a prism 11b, and the first Littrow prism 10a appears to be a prism 15a. The prism 11b is symmetrical to the second Littrow prism 10b with respect to a plane including the face 10b1. The prism 15a is symmetrical to the first Littrow prism 10a with respect to the plane including the face 10b1. The face 10a1 as a passing face appears to be a corresponding face 15a1 of the prism 15a.

Therefore, the second light beam Lb corresponds to an apparent light beam Ld that passes from the apparent subject through the actual second Littrow prism 10b and through a thick plate combining the two apparent prisms 11b and 15a. The two-dot chain arrow in FIG. 16 represents the apparent light beam Ld. When there is the actual second light beam Lb passing through the second Littrow prism 10b, the apparent light beam Ld can be considered. On the other hand, when the apparent light beam Ld cannot be considered, there is no actual second light beam Lb.

The presence of a light beam passing through the face 15a1 of the apparent prism 15a and the face 10b3 of the second Littrow prism 10b is a prerequisite for imaging of the second light beam Lb.

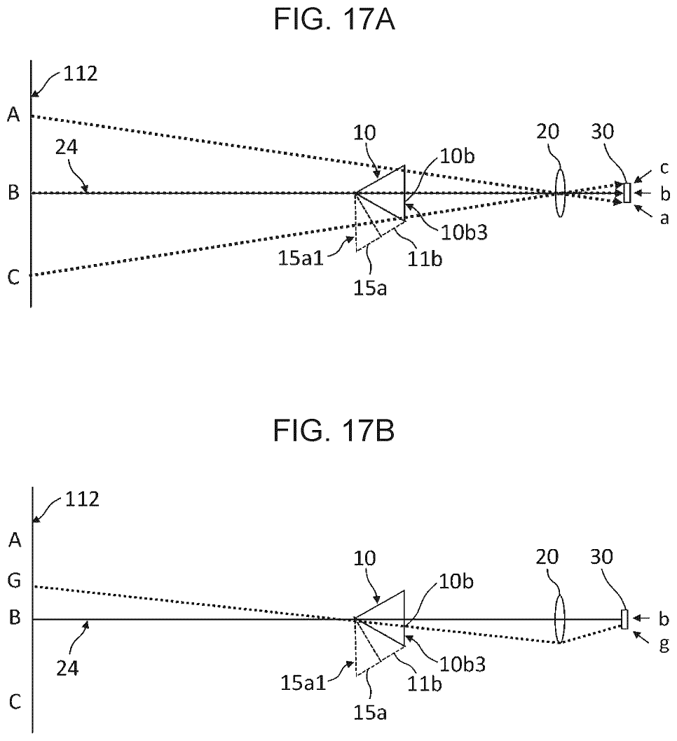

FIGS. 17A and 17B are diagrams each schematically illustrating how a light ray emitted from a certain point is imaged on the image sensor 30 when there are the apparent prisms 11b and 15a, in addition to the dichroic prism 10, for the second light beam Lb.

As illustrated in FIG. 17A, a line segment BC is located on the side of the faces 15a1 and 10b3 through which the second light beam Lb is to pass, with respect to the plane 24. Therefore, a point on the line segment BC on the apparent subject can be imaged in the imaging range of the image sensor 30 by appropriately designing the size and positions of the first Littrow prism 10a and the second Littrow prism 10b.

On the other hand, a line segment AB is located on the opposite side to the faces 15a1 and 10b3 with respect to the plane 24. Therefore, a principal ray emitted from a point on the line segment AB does not pass through the faces 15a1 and 10b3, regardless of the size and positions of the first Littrow prism 10a and the second Littrow prism 10b. However, a light ray that has passed through an optical path other than the principal ray may be imaged in the imaging range of the image sensor 30 even if the light ray is emitted from the point on the line segment AB.

As illustrated in FIG. 17B, a point G is the apparent position that is the boundary of the range where the light ray that has passed through the faces 15a1 and 10b3 is imaged in the imaging range on the image sensor 30 by the imaging optical system 20. A point g is the position where the light ray that appears to be emitted from the point G is imaged on the image sensor 30.

A point on a line segment G-B on the apparent subject is imaged on a line segment g-b in the imaging range of the image sensor 30 by the imaging optical system 20. A point on a line segment A-G on the apparent subject is not imaged in the imaging range on the image sensor 30 because a principal ray emitted from the point does not pass through the imaging optical system 20.

If no dichroic prism 10 is disposed, the range of a line segment A-C on the subject is imaged in the imaging range of the image sensor 30. On the other hand, if the dichroic prism 10 is actually disposed, only the range of a line segment B-C and the line segment G-B on the apparent subject is imaged in the imaging range of the image sensor 30 for the second light beam Lb. The range of the line segment A-G on the apparent subject is not imaged because no light ray reaches the image sensor 30.

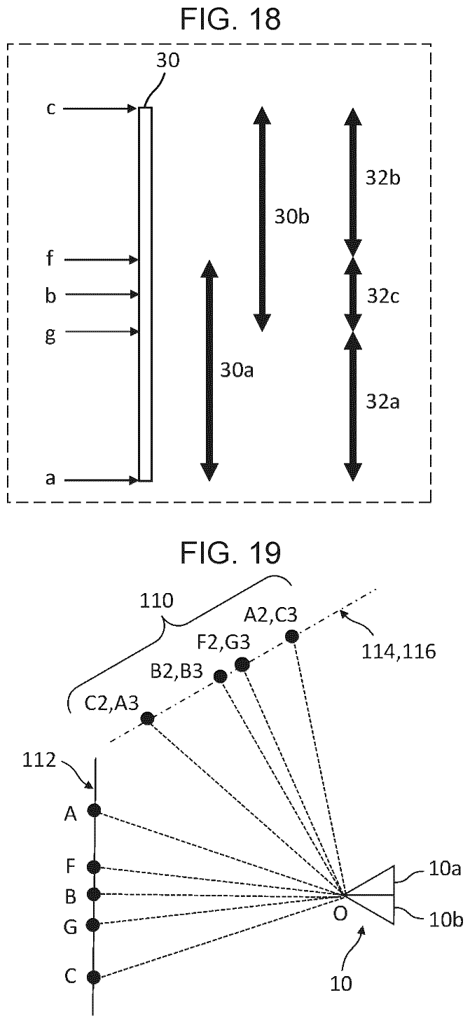

FIG. 18 is a diagram schematically illustrating the range in which the first light beam La and the second light beam Lb are imaged on the image sensor 30. As illustrated in FIG. 18, a first image is formed in a range 30a of a line segment a-f on the image sensor 30, and a second image is formed in a range 30b of a line segment g-c. In this case, only the first image is formed in a range 32a of a line segment a-g, only the second image is formed in a range 32b of a line segment f-c, and the first and second images are formed overlapping each other in a range 32c of a line segment g-f.

### Positional Relationship Between Apparent Subject and Actual Subject

FIG. 19 is a diagram for explaining the positional relationship between the apparent subject and the actual subject. As illustrated in FIG. 19, for the first image, the points A, B, and F of the apparent subject correspond to the points A2, B2, and F2 of the actual subject, respectively. Similarly, for the second image, the points B, C, and G of the apparent subject correspond to the points B3, C3, and G3 of the actual subject, respectively. As described above, the points A2 and C3 are identical, and the points B2 and B3 are identical. Similarly, the points F2 and G3 are identical.

### Positional Relationship Between First and Second Images

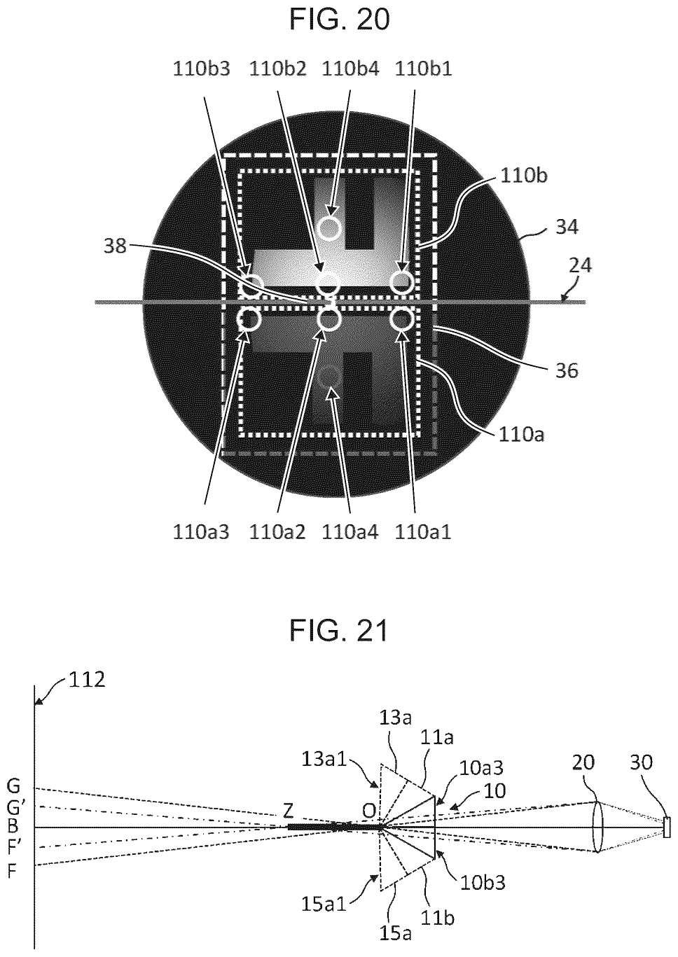

FIG. 20 is a diagram schematically illustrating an example of first and second images formed on the image sensor 30. The subject 110 illustrated in FIG. 3 is as described above with reference to FIG. 45.

There is an imaging range 36 of the image sensor 30 within an image circle 34 formed on the image sensor 30 by the imaging optical system 20. A first image 110a and a second image 110b are formed in the imaging range 36. The first image 110a and the second image 110b are in a mirror-image symmetry relationship with respect to the plane 24. The first image 110a and the second image 110b are formed in the ranges 32a and 32b illustrated in FIG. 18 so as not to overlap with each other. The range 32a corresponds to the range of the line segment A2-F2 illustrated in FIG. 19, and the range 32b corresponds to the range of the line segment G3-C3 illustrated in FIG. 19.

The first light beam La and the second light beam Lb emitted from a certain point in the subject 110 and imaged on the image sensor 30 travel along the same optical path until they reach the face 10a2. After reaching the face 10a2, the first light beam La and the second light beam Lb travel along different optical paths that are symmetrical with respect to the plane 24. These different optical paths pass through the dichroic prism 10 and the imaging optical system 20.

In the first light beam La and the second light beam Lb traveling along such symmetrical optical paths, vignetting is caused to the same extent by the dichroic prism 10 and the imaging optical system 20. Peripheral darkening is also caused to the same extent by the dichroic prism 10 and the imaging optical system 20. Therefore, in the first image 110a and the second image 110b, light rays having wavelengths included in the first and second wavelength ranges emitted from the same point in the subject 110 have the same degree of darkening due to vignetting and peripheral darkening. By calculating an intensity ratio thereof, the effects of vignetting and peripheral darkening can be ignored.

The first image 110a and the second image 110b may be both distorted by curvature aberration. However, the curvature aberration occurs symmetrically with respect to the plane 24. Corresponding positions in the first image 110a and the second image 110b are symmetric with respect to the plane 24, regardless of the shape of the curvature aberration. Therefore, it is very easy to find the corresponding positions.

As illustrated in FIG. 20, a point 110b1 in the second image 110b corresponds to a point 110a1 in the first image 110a. Similarly, a point 110b2 in the second image 110b corresponds to a point 110a2 in the first image 110a. A point 110b3 in the second image 110b corresponds to a point 110a3 in the first image 110a. A point 110b4 in the second image 110b corresponds to a point 110a4 in the first image 110a. The points 110b1 to 110b4 in the second image 110b are symmetrical to the points 110a1 to 110a4 in the first image 110a, respectively, with respect to the plane 24. The respective points corresponding to the same position in the subject 110 have the same distance from a reference position 38. The reference position 38 is the position where the optical axis of the imaging optical system 20 passes through the imaging range 36.

As described above, the first image 110a and the second image 110b are in a mirror-image symmetry relationship with respect to the plane 24. A ratio of radiation intensities from corresponding points in the first image 110a and the second image 110b is calculated to correct the sensitivity of the image sensor 30 as necessary. As a result, the ratio of radiation intensities of the first and second wavelength ranges from corresponding points in the subject 110 can be obtained accurately.

### Measures to Reduce Overlapping Range of First and Second Images

The range of the line segment F-G illustrated in FIG. 19 corresponds to the overlapping range of the first image 110a and the second image 110b on the image sensor 30. The first image 110a and the second image 110b can be obtained separately by performing a mathematical separation operation. However, it is more practical to obtain the first image 110a and the second image 110b separately without such an operation.

A first method for narrowing the overlapping range of the first image 110a and the second image 110b will be described. The first method is using a light shielding body. FIG. 21 is a diagram schematically illustrating how a light ray emitted from a certain point is imaged on the image sensor 30 when there are apparent prisms 11a, 13a, 15a, and 11b in addition to the dichroic prism 10 for the first light beam La and the second light beam Lb. As illustrated in FIG. 21, a point F′ is an apparent position where a light ray passing through a point Z, a face 13a1, and a face 10a3 is imaged in the imaging range on the image sensor 30 by the imaging optical system 20. Similarly, a point G′ is an apparent position where a light ray passing through the point Z, a face 15al, and a face 10b3 is imaged in the imaging range on the image sensor 30 by the imaging optical system 20. The point Z is located on a line segment O-B.

As illustrated in FIG. 21, an apparent light shielding body represented by a thick line is located between the points O and Z. A light ray that crosses the light shielding body is not imaged on the image sensor 30. A light ray emitted from the line segment A-B illustrated in FIG. 19 and imaged on the image sensor 30 does not cross the apparent light shielding body. Therefore, the apparent light shielding body does not affect a range in which a principal ray having a wavelength included in the first wavelength range is imaged on the image sensor 30.

On the other hand, the apparent light shielding body blocks the light ray having the wavelength included in the first wavelength range, which is emitted from a point on an apparent line segment F-F′ and imaged on the image sensor 30. Similarly, the apparent light shielding body blocks a light ray having the wavelength included in the second wavelength range, which is emitted from a point on an apparent line segment G-G′ and imaged on the image sensor 30.

f′ represents a point on the image sensor 30 that is conjugate with the apparent position F′. g′ represents a point on the image sensor 30 that is conjugate with the apparent position G′. The point f′ is located between the points b and f illustrated in FIG. 18. The point g′ is located between the points g and b illustrated in FIG. 18. In this case, the overlapping range of the first image 110a and the second image 110b on the image sensor 30 is the range of a line segment f′-g′. Therefore, the overlapping range of the first image 110a and the second image 110b is narrowed.

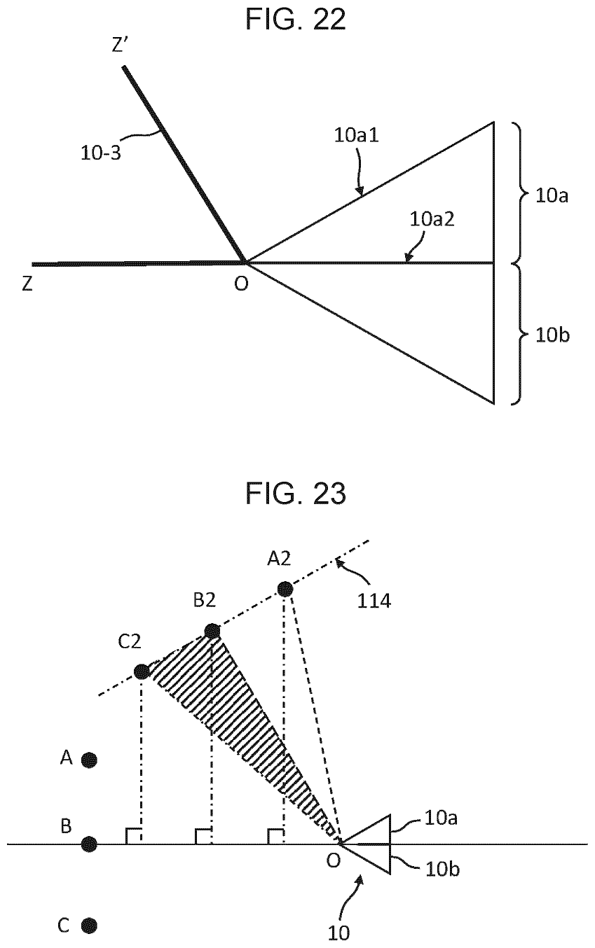

FIG. 22 is a diagram schematically illustrating a positional relationship between an apparent light shielding body, an actual light shielding body, and the dichroic prism 10. As illustrated in FIG. 22, an actual light shielding body 10-3 is located between a point O and a point Z′, where Z′ is an actual point corresponding to an apparent point Z. The point Z′ is located on the line segment O-B2 or line segment O-B3 illustrated in FIG. 19.

FIG. 23 is a diagram schematically illustrating a range of blocking the optical path along which the first light beam La and the second light beam Lb are both imaged on the image sensor 30. By disposing the light shielding body 10-3 in the range of the hatched triangle OB2C2 illustrated in FIG. 23, the overlapping range of the first image 110a and the second image 110b on the image sensor 30 can be reduced.

When the optical path adjusting element 10-1 is disposed as illustrated in FIG. 3, the light shielding body 10-3 is disposed at a position between the optical path adjusting element 10-1 and the subject 110 where the optical path is blocked, along which the first light beam La and the second light beam Lb are both imaged on the image sensor 30.

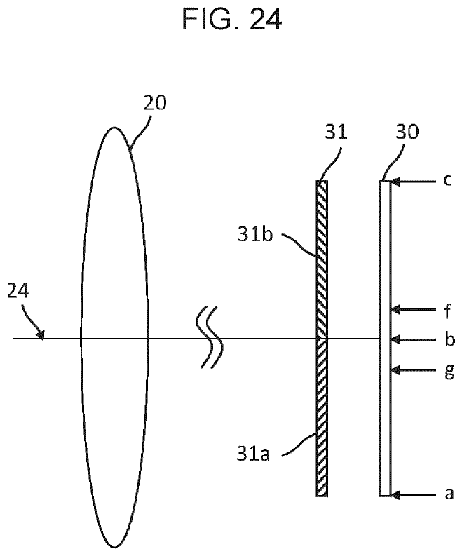

Next, a second method for narrowing the overlapping range of the first image 110a and the second image 110b will be described. The second method is using a filter array. FIG. 24 is a diagram schematically illustrating an example of a filter array 31. As illustrated in FIG. 24, the filter array 31 is disposed between the imaging optical system 20 and the image sensor 30. The filter array 31 includes a first filter portion 31a and a second filter portion 31b. The first filter portion 31a transmits the first light beam La and blocks the second light beam Lb by reflection or absorption. The second filter portion 31b transmits the second light beam Lb and blocks the first light beam La by reflection or absorption.

The first filter portion 31a is disposed above a first region corresponding to a line segment a-b on the image sensor 30, where the term “above” means a direction perpendicular to the imaging surface of the image sensor 30 and away from the imaging surface. The second filter portion 31b is disposed above a second region corresponding to a line segment b-c on the image sensor 30. Therefore, in the image sensor 30, a first image 110a can be obtained in the first region, and a second image 110b can be obtained in the second region. When the filter array 31 is disposed directly above the image sensor 30, the plurality of photoelectric conversion elements included in the image sensor 30 all receive light having a wavelength included in either one of the first and second wavelength ranges.

When the filter array 31 is disposed away from the image sensor 30, some of the plurality of photoelectric conversion elements included in the image sensor 30 can receive light having a wavelength included in the first wavelength range and light having a wavelength included in the second wavelength range, due to the influence of light that passes obliquely through the first filter portion 31a or the second filter portion 31b. The number of these some photoelectric conversion elements can be reduced by the following method, for example. The method is limiting the distance between the filter array 31 and the image sensor 30 relative to the size of the image sensor 30. Alternatively, the method is improving telecentric characteristics of the imaging optical system 20.

The filter array 31 includes a single first filter portion 31a and a single second filter portion 31b. Therefore, the filter array 31 is easier to fabricate than a configuration in which a plurality of filters are formed corresponding to the plurality of photoelectric conversion elements. In the configuration in which a plurality of filters are formed corresponding to the plurality of photoelectric conversion elements, the positional relationship between each photoelectric conversion element and the filter corresponding thereto is designed to sufficiently reduce positional displacement therebetween relative to the size of each photoelectric conversion element. On the other hand, the positional relationship between the first region and the first filter portion 31a is less affected by the positional displacement therebetween, and thus does not need to be designed very strictly. The same holds true for the positional relationship between the second region and the second filter portion 31b.

### Additional Description of Optical Path Adjusting Element 10-1

As illustrated in FIG. 19, the actual subject 110 is not located on the extension of the optical axis of the imaging optical system 20, unlike the apparent subject. Therefore, the orientation of the imaging optical system 20 is significantly different between the case where the dichroic prism 10 is disposed and the case where the dichroic prism 10 is not disposed. This problem can be solved by disposing the optical path adjusting element 10-1 between the subject 110 and the dichroic prism 10. The optical path adjusting element 10-1 allows the imaging optical system 20 to be oriented in a natural direction.

The natural direction of the imaging optical system 20 is, for example, a direction in which the first image 110a and the second image 110b are formed on the image sensor 30 so as not to overlap with each other, and a direction in which the central axis of the area between the first image 110a and the second image 110b intersects the subject 110. When this direction coincides with the optical axis of the imaging optical system 20, the subject 110 is located in the optical axis direction of the imaging optical system 20 and faces the image sensor 30. In that case, the first image 110a and the second image 110b of the subject 110 located in that direction can be formed on the image sensor 30 so as not to overlap with each other.

When the apparent subject is within the range of the line segment A-B, the optical path adjusting element 10-1 can be designed, for example, so that the light beam from the midpoint of the line segment A-B is parallel to the optical axis of the imaging optical system 20.

The imaging device 100 may further include an adjusting tool for adjusting the positions and orientations of the optical path adjusting element 10-1 and the dichroic prism 10. Alternatively, when the optical path adjusting element 10-1 includes a plurality of mirrors or prisms, the imaging device 100 may further include an adjusting tool for adjusting the positions and orientations of a plurality of components included in the optical path adjusting element 10-1.

By replacing a lens and operating a zoom lens, the focal length of the imaging optical system 20 can be changed, causing a change in the range of the subject image on the image sensor 30. This can cause a change in the center of the imaging range on the imaging surface of the image sensor 30. Even in such a case, the adjusting tool described above can be used to adjust the optical axis of the imaging optical system 20 so that it passes through the center of the imaging range in a direction perpendicular to the imaging surface of the image sensor 30.

### Arrangement of Dichroic Prism 10

The size of the image sensor 30 is usually several mm to several tens of mm. Therefore, the area of the subject 110 is often larger than the imaging surface of the image sensor 30. Therefore, the first and second images formed on the image sensor 30, more specifically on the imaging surface thereof, is each smaller than the subject 110. When imaging an area of the subject 110 larger than the imaging surface of the image sensor 30, the spread angle of the light beam formed by the imaging optical system 20 is smaller on the subject 110 side and larger on the image sensor 30 side.

In the imaging device 100, the dichroic prism 10 is arranged on the subject 110 side with respect to the imaging optical system 20. This makes it possible to reduce the aberration caused when the light beam having the spread angle passes through the dichroic prism 10, compared to a configuration in which the dichroic prism 10 is arranged on the image sensor 30 side.

### Connection of Optical Component 10A and Lens Unit 20A

In the imaging device 100, a general commercially available lens unit and a general commercially available camera can be used as the lens unit 20A and the camera 30A. Hereinafter, description will be given of an example of connection of the optical component 10A and the lens unit 20A to facilitate the use of the commercially available lens unit and the commercially available camera. The optical component 10A is detachable from the lens unit 20A and can be connected to the commercially available lens unit.

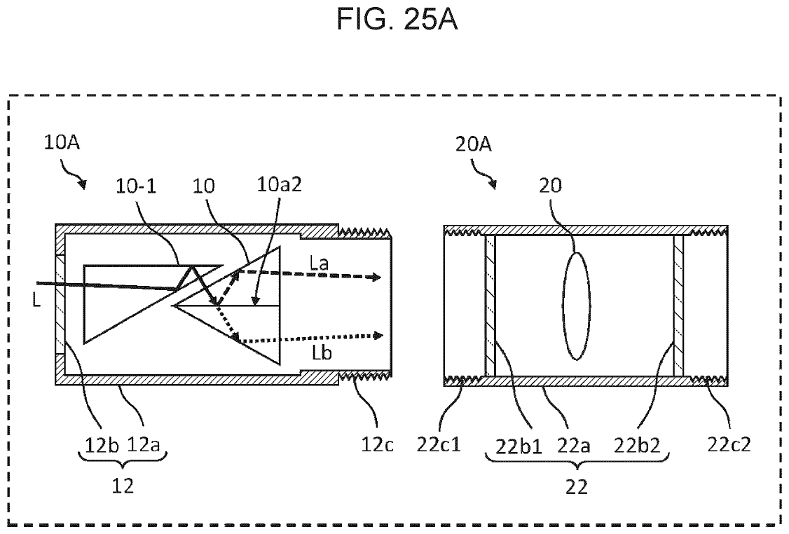

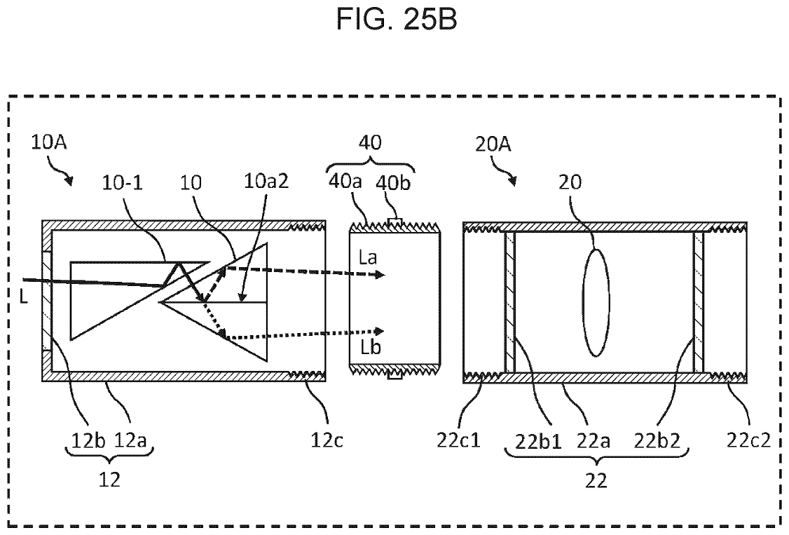

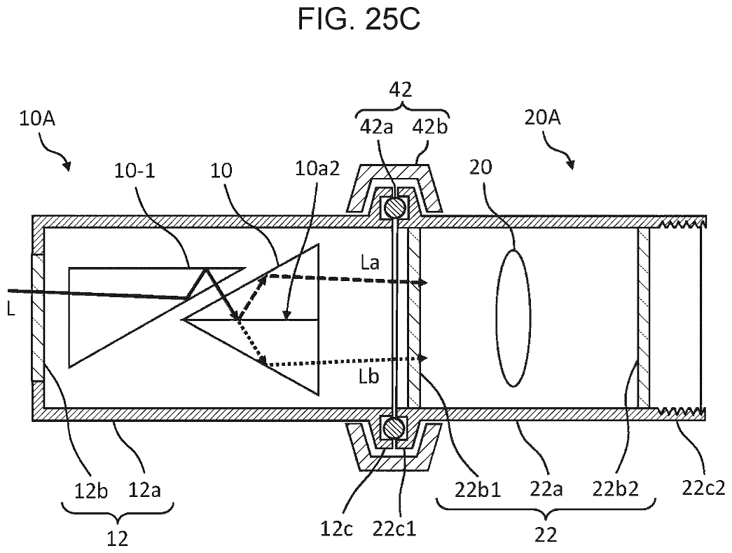

FIG. 25A is a diagram schematically illustrating an example of connection of the optical component 10A and the lens unit 20A. The optical component 10A includes the dichroic prism 10, the optical path adjusting element 10-1, and the housing 12 that houses them. The housing 12 includes a side wall 12a and a light transmitting window 12b. The side wall 12a surrounds and supports the dichroic prism 10 and the optical path adjusting element 10-1. The side wall 12a has a light shielding property and may function as the light shielding body 10-2 illustrated in FIG. 3. The light transmitting window 12b transmits the light beam L from the subject 110. The light transmitting window 12b is located near the front end of the side wall 12a. The light transmitting window 12b may be, for example, an optical filter that is transparent to the first and second wavelength ranges and blocks or attenuates light beams having wavelengths included in wavelength ranges other than the first and second wavelength ranges. Alternatively, the light transmitting window 12b may be omitted.

The housing 12 further includes a connection structure 12c located near the rear end of the side wall 12a. The connection structure 12c is a structure for connecting the optical component 10A and the lens unit 20A. The connection structure 12c may be, for example, a male screw.

The lens unit 20A includes the imaging optical system 20 and the lens housing 22 that houses the imaging optical system 20. The lens housing 22 may include a side wall 22a, a light transmitting window 22b1, and a light transmitting window 22b2. The side wall 22a surrounds and supports the imaging optical system 20. The light transmitting window 22b1 and the light transmitting window 22b2 transmit the first light beam La and the second light beam Lb. The light transmitting window 22b1 is located near the front end of the side wall 22a, and the light transmitting window 22b2 is located near the rear end of the side wall 22a. The light transmitting windows 22b1 and 22b2 may be made of a light transmitting material such as glass, or may simply be hollow.

The lens housing 22 further includes a connection structure 22cl located near the front end of the side wall 22a, and a connection structure 22c2 located near the rear end of the side wall 22a. The connection structure 22cl is a structure for attaching a filter of a camera lens. The connection structure 22c2 is a structure for connecting the lens unit 20A and the camera 30A. The connection structures 22c1 and 22c2 can be, for example, female screws.

The optical component 10A and the lens unit 20A can be connected by the connection structure 12c of the optical component 10A and the connection structure 22c1 of the lens unit 20A. The central axis of the connection structure 12c in the optical component 10A is located on the optical axis of the imaging optical system 20. The dichroic prism 10 can be fixed in the housing 12 so that the central axis is included in the face 10a2 of the dichroic prism 10, for example. In that case, by connecting the optical component 10A and the lens unit 20A, the plane including the face 10a2 can include the optical axis of the imaging optical system 20.

FIG. 25B is a diagram schematically illustrating another example of connecting the optical component 10A and the lens unit 20A. The imaging device 100 illustrated in FIG. 3 may further include an adjusting tool 40 illustrated in FIG. 25B. The adjusting tool 40 adjusts the positional relationship between the dichroic prism 10 and the imaging optical system 20. The positional relationship therebetween may be, for example, a relative angle therebetween. The optical component 10A and the imaging optical system 20 are connected using the adjusting tool 40. The connection structure 12c of the optical component 10A may be, for example, a female screw. The relative angle may be adjusted, for example, by adjusting the amount of screwing of the female screw.

The adjusting tool 40 has a generally cylindrical shape. The adjusting tool 40 may include, for example, a full-thread screw 40a and a tightening ring 40b. The front end of the full-thread screw 40a is connected to the female screw near the rear end of the optical component 10A, and the rear end of the full-thread screw 40a is connected to the female screw near the front end of the lens unit 20A. After the front end of the full-thread screw 40a is screwed into the female screw near the rear end of the optical component 10A to a desired position, the optical component 10A and the lens unit 20A are fixed by the tightening ring 40b. This makes it possible to adjust the positional relationship between the dichroic prism 10 and the imaging optical system 20 to a desired positional relationship, more specifically, a desired angular relationship.

FIG. 25C is a diagram schematically illustrating yet another example of connecting the optical component 10A and the lens unit 20A. As illustrated in FIG. 25C, the connection structure 12c of the optical component 10A and the connection structure 22c1 of the lens unit 20A may have a flange structure similar to an ISO-KF flange, for example. The imaging device 100 illustrated in FIG. 3 may further include a fixing tool 42 illustrated in FIG. 25C. The fixing tool 42 may include, for example, a centering ring 42a and a clamp ring 42b. The centering ring 42a facilitates the alignment of the connection structure 12c of the optical component 10A, which is the flange structure, and the connection structure 22cl of the lens unit 20A. The clamp ring 42b allows the connection between the connection structure 12c of the optical component 10A, which is the flange structure, and the connection structure 22c1 of the lens unit 20A. As a result, the positional relationship between the dichroic prism 10 and the imaging optical system 20 becomes the desired positional relationship, more specifically, the desired angular relationship.

As described above, by connecting the optical component 10A and the lens unit 20A, the dichroic prism 10 and the imaging optical system 20 can be fixed in a desired angular relationship. Furthermore, by connecting the lens unit 20A and the camera 30A, the imaging optical system 20 and the image sensor 30 can be fixed in a desired angular relationship. Therefore, by connecting the optical component 10A, the lens unit 20A, and the camera 30A, the dichroic prism 10 and the image sensor 30 can be fixed in a desired angular relationship. As a result, as illustrated in FIG. 20, the plane 24 can be positioned so as to be parallel to the short side of the rectangular imaging range and pass through the midpoint of the long side.

If the components illustrated in FIGS. 25A to 25C are not used, the phase of the female screw provided in the commercially available lens unit differs for each lens unit, making it difficult to fix the dichroic prism 10 and the image sensor 30 in the desired angular relationship.

As described above, in the imaging device 100 according to Embodiment 1, the first image 110a and the second image 110b are in a mirror-image symmetry relationship. Such a relationship makes it easier to identify the corresponding positions in these two images. The first light beam La and the second light beam Lb travel symmetrically with respect to the plane 24 including the optical axis of the imaging optical system 20, thus causing the curvature aberration and peripheral darkening to occur almost equally in the first and second wavelength ranges. Therefore, the intensity ratio at corresponding positions in the first image 110a and the second image 110b is almost unlikely to be affected by the curvature aberration and peripheral darkening. The first image 110a and the second image 110b are thus suitable for evaluation of the subject 110, for example, in two-color thermography and fluorescence imaging.

Furthermore, in the imaging device 100 according to Embodiment 1, a commercially available lens unit and a commercially available camera can be used as the lens unit 20A and the camera 30A. In the imaging device 100, the optical component 10A is added to such lens unit 20A and camera 30A. The imaging device 100 thus has a simple configuration.

Therefore, the imaging device 100 according to Embodiment 1 can obtain two images in different wavelength ranges suitable for evaluation of the subject 110 with a simple configuration. Furthermore, in the imaging device 100 according to Embodiment 1, the optical path adjusting element 10-1 may be disposed between the subject 110 and the dichroic prism 10. The optical path adjusting element 10-1 allows the subject 110 to be located in the optical axis direction of the imaging optical system 20 and also imaged in a natural direction of facing the image sensor 30.

### Modifications of Imaging Device 100 According to Embodiment 1

Modifications 1 and 2 of the imaging device 100 according to Embodiment 1 will be described below. The first subcomponent 10A1 and the second subcomponent 10A2 illustrated in FIG. 1 are not limited to the dichroic prism 10 and the optical path adjusting element 10-1 illustrated in FIG. 3, respectively.

### Modification 1

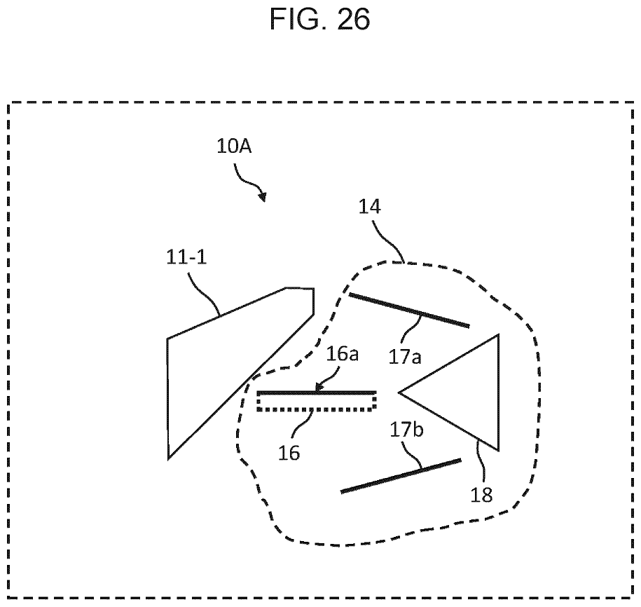

FIG. 26 is a diagram schematically illustrating a specific configuration of the optical component 10A in Modification 1 of the imaging device 100 according to Embodiment 1. An optical component 10A illustrated in FIG. 26 includes an optical element group 14 and an optical path adjusting element 11-1. The optical component 10A illustrated in FIG. 26 may further include the light shielding body 10-2 and the housing 12 illustrated in FIG. 3. The optical element group 14 includes a dichroic mirror 16 having a dichroic surface 16a, two mirrors 17a and 17b, and a triangular prism 18. The optical path adjusting element 11-1 is a half pentaprism. The optical element group 14 corresponds to the first subcomponent 10A1 illustrated in FIG. 1. The optical path adjusting element 11-1 corresponds to the second subcomponent 10A2 illustrated in FIG. 1. In this specification, the optical element group 14 is also simply referred to as a “first optical element”, and the optical path adjusting element 11-1 is also referred to as a “second optical element”.

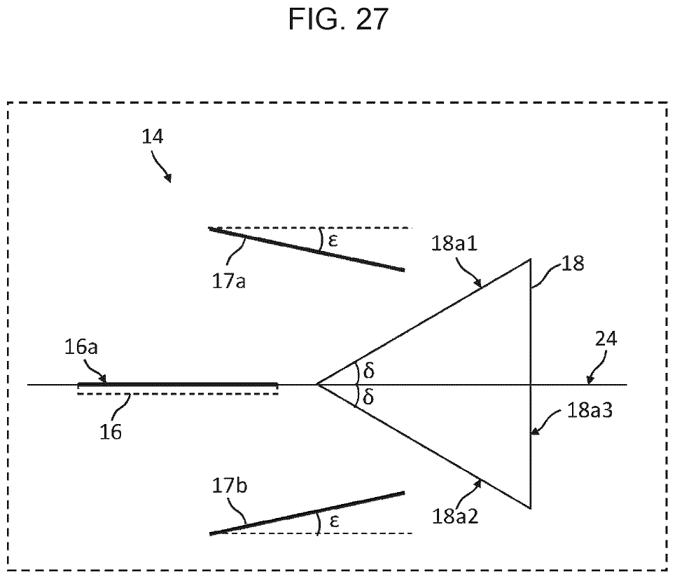

FIG. 27 is a diagram schematically illustrating the optical element group 14 according to Modification 1. As illustrated in FIG. 27, the dichroic mirror 16 is a flat optical element having the dichroic surface 16a. The dichroic surface 16a is arranged in the plane 24. The two mirrors 17a and 17b are arranged symmetrically with respect to the plane 24. The mirror 17a and the plane 24 form an angle &. The same is true for an angle formed by the mirror 17b and the plane 24. However, the mirrors 17a and 17b are tilted in opposite directions.

The triangular prism 18 has a triangular prism shape, and has a face 18a1, a face 18a2, and a face 18a3 as its side faces. The faces 18al and 18a2 are arranged symmetrically with respect to the plane 24. The face 18al and the plane 24 form an angle δ. The same is true for an angle formed by the face 18a2 and the plane 24. However, the faces 18a1 and 18a2 are tilted in opposite directions.

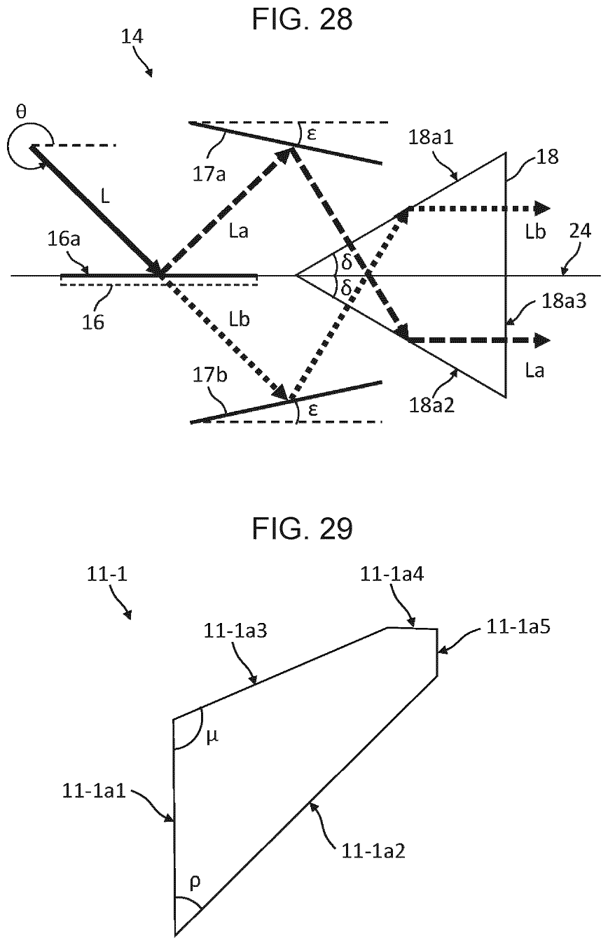

FIG. 28 is a diagram schematically illustrating an example of the paths of a light beam L, a first light beam La, and a second light beam Lb within the optical element group 14 according to Modification 1. As illustrated in FIG. 28, the dichroic surface 16a of the dichroic mirror 16 reflects the first light beam La of the light beam L, which is incident in the direction θ, in the direction −θ, and transmits the second light beam Lb in the direction θ. The dichroic surface 16a thus separates the light beam L into the first light beam La and the second light beam Lb.

The mirror 17a reflects the first light beam La to make it incident on the triangular prism 18, and the mirror 17b reflects the second light beam Lb to make it incident on the triangular prism 18. The traveling direction of the first light beam La reflected by the mirror 17a is θ−2ε, and the traveling direction of the second light beam Lb reflected by the mirror 17b is −θ+2ε.

The triangular prism 18 emits the incident first light beam La and second light beam Lb to the outside of the triangular prism 18. The first light beam La is incident on the face 18a1, totally reflected on the face 18a2, and emitted to the outside of the triangular prism 18 from the face 18a3. The traveling direction of the first light beam La emitted to the outside is −θ+2ε−2δ. The second light beam Lb is incident on the face 18a2, totally reflected on the face 18a1, and emitted to the outside of the triangular prism 18 from the face 18a3. The traveling direction of the second light beam Lb emitted to the outside is θ−2ε+2δ. The first light beam La and the second light beam Lb are symmetric with respect to the plane 24.

As described above, the optical element group 14 separates the light beam L into the first light beam La and the second light beam Lb by the dichroic surface 16a. The optical element group 14 also converts the directions of the first light beam La and the second light beam Lb by the two mirrors 17a, 17b and the triangular prism 18 and emits them.

It is assumed that the light beam L is incident on the dichroic surface 16a in a direction θ=−π/4. When the angle ε is π/24, the first light beam La is incident on the face 18al in a direction −π/3, and the second light beam Lb is incident on the surface 18a2 in a direction π/3. Furthermore, when the angle δ is about π/6, the traveling directions of the first light beam La and the second light beam Lb emitted to the outside become nearly zero.

FIG. 29 is a diagram schematically illustrating the optical path adjusting element 11-1 according to Modification 1. As illustrated in FIG. 29, the optical path adjusting element 11-1 has a face 11-1a1, a face 11-1a2, and a face 11-1a3. The optical path adjusting element 11-1 further has a face 11-1a4 and a face 11-1a5. The faces 11-1a4 and 11-1a5 are faces through which the light beam L does not pass, and may be omitted.

The faces 11-1a1 and 11-1a2 form an angle ρ. The faces 11-1a1 and 11-1a3 form an angle μ. The face 11-1a3 may have a metal film or a dielectric multilayer film to achieve a high light reflectivity.

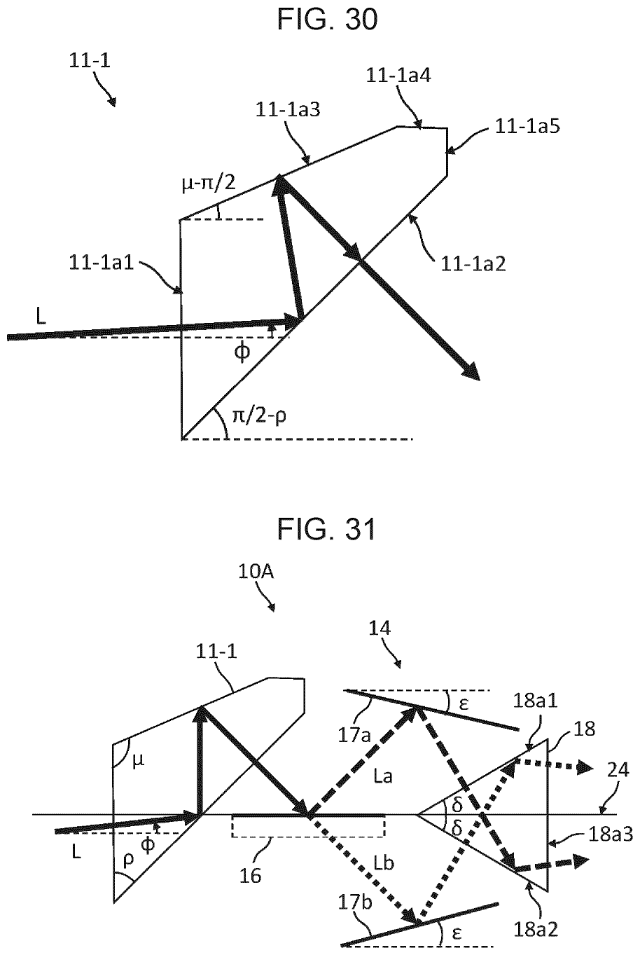

FIG. 30 is a diagram schematically illustrating an example of the path of the light beam L in the optical path adjusting element 11-1 according to Modification 1. In the example illustrated in FIG. 30, it is assumed that the face 11-1a1 is perpendicular to the plane 24 illustrated in FIG. 27. In the optical path adjusting element 11-1, the light beam L is reflected twice inside. Therefore, as long as the light beam L passes through the incident surface and the reflecting surface in the order described below, the orientation of the optical path adjusting element 11-1 does not affect the change in the path of the light beam L.

The light beam L is incident on the face 11-1a1. The traveling direction of the light beam L incident on the face 11-1a1 is q. The light beam L incident on the face 11-1a1 is totally reflected by the face 11-1a2. The traveling direction of the light beam L totally reflected by the face 11-1a2 is −φ+π−2ρ. The light beam L totally reflected by the face 11-1a2 is totally reflected by the face 11-1a3 and emitted to the outside from the face 11-1a2. The traveling direction of the light beam L emitted to the outside from the face 11-1a2 is φ+2ρ+2μ. However, the fact that the angle is the same regardless of the difference of ±2π is used.

If ρ is about π/4 and μ is about 5π/8, 2ρ+2μ is about −π/4. Therefore, the optical path adjusting element 11-1 has a function to change the traveling direction of the light beam L by about −π/4.

FIG. 31 is a diagram schematically illustrating an example of the paths of the light beam L, the first light beam La, and the second light beam Lb within the optical component 10A according to Modification 1. As illustrated in FIG. 31, the light beam L incident on the optical path adjusting element 11-1 in the direction φ is reflected inside and then emitted to the outside. The traveling direction of the light beam L emitted to the outside from the optical path adjusting element 11-1 is φ+2ρ+2μ.

The light beam L emitted to the outside from the optical path adjusting element 11-1 is separated into the first light beam La and the second light beam Lb by the dichroic mirror 16. The first light beam La is reflected by the mirror 17a and the face 18a2, and then emitted to the outside from the face 18a3. The second light beam Lb is reflected by the mirror 17b and the face 18a1, and then emitted to the outside from the face 18a3.

The traveling direction of the first light beam La emitted to the outside from the face 18a3 is −φ−2ρ−2μ+2ε−2δ. The traveling direction of the second light beam Lb emitted to the outside from the face 18a3 is φ+2ρ+2μ−2ε+2δ. Therefore, the first light beam La and the second light beam Lb are in a mirror-image symmetry relationship with respect to the plane 24.

The orientation of the faces of the optical path adjusting element 11-1, the orientation of the two mirrors 17a and 17b, and the orientation of the faces of the triangular prism 18 may be set so that 2ρ+2μ−2ε+2δ is nearly zero. With such setting, when the traveling direction of the light beam L incident on the optical path adjusting element 11-1 is zero, the traveling directions of the first light beam La and the second light beam Lb become nearly zero. However, as in Embodiment 1, the traveling directions of the first light beam La and the second light beam Lb do not have to be completely zero. As in Embodiment 1, even if the traveling direction of the light beam L incident on the optical path adjusting element 11-1 is nearly zero, an angular difference can be imparted to the traveling directions of the first light beam La and the second light beam Lb. When the first light beam La and the second light beam Lb travel while approaching each other, the first and second images that are mirror-image symmetric with respect to the plane 24 can be formed at different positions on the image sensor 30.

ρ, μ, and δ of 2ρ+2μ−2ε+2δ are fixed by the shapes of the optical path adjusting element 11-1 and the triangular prism 18, while ε depends on the orientation of the two mirrors 17a and 17b. Therefore, ε can be easily adjusted.

By changing ε, the positions of the first and second images on the image sensor 30 can be moved. Therefore, the positions of the first and second images on the image sensor 30 can be set to desired positions according to the size of the subject 110, the focal length of the imaging optical system 20, and the size of the image sensor 30.

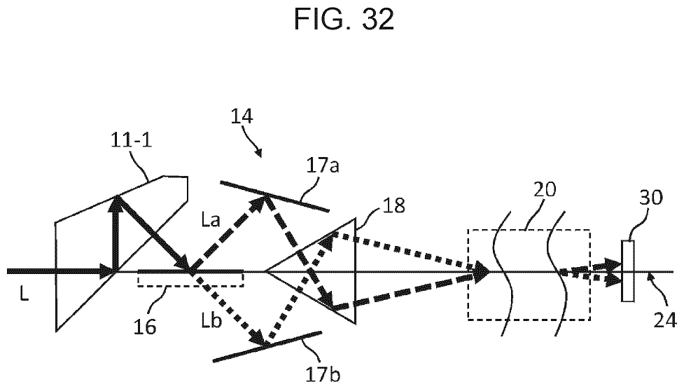

FIG. 32 is a diagram schematically illustrating an example of the paths of the light beam L, the first light beam La, and the second light beam Lb in Modification 1 of the imaging device. As in Embodiment 1, the first and second images can be formed on the image sensor 30 by combining the optical element group 14 and the optical path adjusting element 11-1 according to Modification 1 with the imaging optical system 20, as illustrated in FIG. 32.

In Modification 1, the dichroic mirror 16 is commercially available and can be easily obtained. By replacing the dichroic mirror 16, the first and second wavelength ranges can be easily changed.

Furthermore, in Modification 1, the incident angle at which the light beam L is incident on the dichroic surface 16a can be π/4, for example, where the incident angle is the angle formed by the light beam L and the dichroic surface 16a. Therefore, compared to Embodiment 1 in which the incident angle is as steep as π/3, the dielectric multilayer film constituting the dichroic surface 16a is easier to design and manufacture.

In Modification 1, the positions of the first and second images on the image sensor 30 can also be adjusted by changing the orientation of the two mirrors 17a and 17b.

### Modification 2

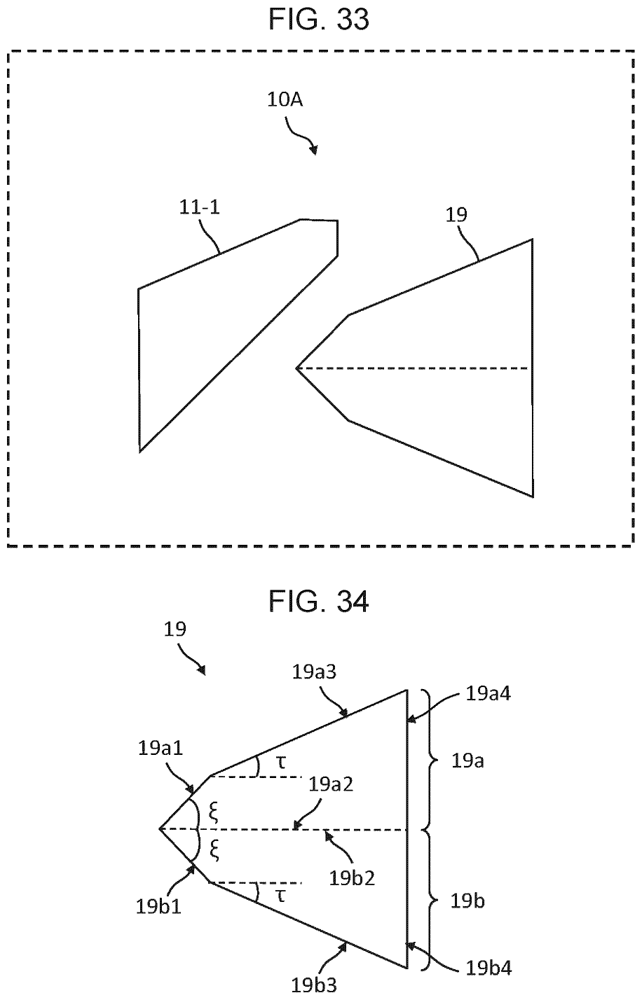

FIG. 33 is a diagram schematically illustrating a specific configuration of an optical component 10A according to Modification 2 of the imaging device 100 according to Embodiment 1. The optical component 10A illustrated in FIG. 33 includes a dichroic prism 19 and an optical path adjusting element 11-1. The optical component 10A illustrated in FIG. 33 may further include the light shielding body 10-2 and the housing 12 illustrated in FIG. 3. The dichroic prism 19 corresponds to the first subcomponent 10A1 illustrated in FIG. 1. The optical path adjusting element 11-1 corresponds to the second subcomponent 10A2 illustrated in FIG. 1. In this specification, the dichroic prism 19 is also referred to as a “first optical element”.

FIG. 34 is a diagram schematically illustrating the dichroic prism 19 according to Modification 2. As illustrated in FIG. 34, the dichroic prism 19 includes a first prism 19a and a second prism 19b. The first prism 19a has a face 19a1, a face 19a2, a face 19a3, and a face 19a4. The second prism 19b has a face 19b1, a face 19b2, a face 19b3, and a face 19b4.

In the first prism 19a, the face 19al and the face 19a2 form an angle ξ, and the face 19a2 and the face 19a3 form an angle t. The face 19a2 and the face 19a4 form an angle π/2. In the second prism 19b, the face 19b1 and the face 19b2 form an angle ξ, and the face 19b2 and the face 19b3 form an angle t. The face 19b2 and the face 19b4 form an angle π/2.

The face 19a2 and the face 19b2 are bonded with an adhesive layer. The adhesive layer is translucent in the second wavelength range. The face 19a2 and the face 19b2 may be parallel to each other, for example. As a result of bonding the face 19a2 and the face 19b2, the first prism 19a and the second prism 19b are arranged so as to be mirror-image symmetrical to each other.

One or both of the faces 19a2 and 19b2 may be dichroic surfaces that reflect a first light beam La and transmit a second light beam Lb of a light beam L incident from a direction in a specific range, for example. The dichroic surface has the above function in a state where the first prism 19a and the second prism 19b are bonded together. Here, the direction in the specific range refers to a direction in a range where the light beam L from the subject 110 is incident. The dichroic prism 19 thus has at least one dichroic surface as described above. The faces 19a3 and 19b3 may each have a metal film or a dielectric multilayer film to achieve a high light reflectivity.

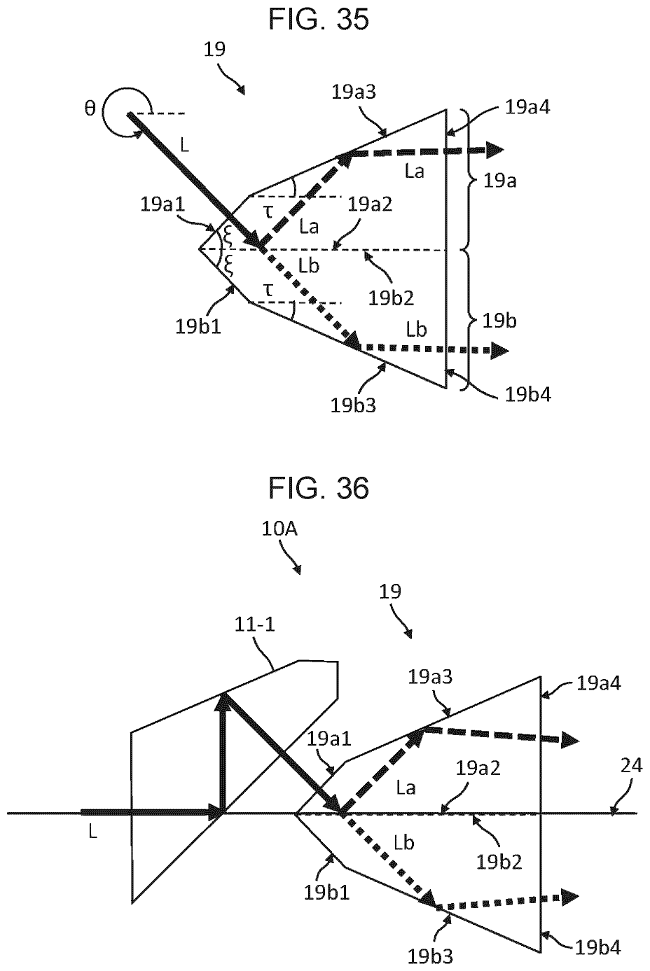

FIG. 35 is a diagram schematically illustrating an example of the paths of the light beam L, the first light beam La, and the second light beam Lb in the dichroic prism 19 according to Modification 2. As illustrated in FIG. 35, the face 19a2 of the dichroic prism 19 reflects the first light beam La of the light beam L, which is incident in the direction θ, in the direction −θ, and transmits the second light beam Lb in the direction θ. In the first prism 19a, the first light beam La is totally reflected by the face 19a3 and emitted to the outside from the face 19a4. The traveling direction of the first light beam La emitted to the outside is θ+2ξ+2τ. In the second prism 19b, the second light beam Lb is totally reflected by the face 19b3 and emitted to the outside from the face 19b4. The traveling direction of the second light beam Lb emitted to the outside is −θ−2ξ−2τ.

As described above, the dichroic prism 19 separates the light beam L into the first light beam La and the second light beam Lb by the face 19a2. The dichroic prism 19 also converts the directions of the first light beam La and the second light beam Lb by the faces 19a3 and 19b3 and emits them.

When θ is about −π/4, ξ is about π/4, and τ is about (7/8)π, the traveling directions of the first light beam La and the second light beam Lb emitted to the outside become nearly zero. Since θ is about −π/4 and ξ is about π/4, the light beam L can be made incident almost perpendicularly on the face 19a1, making it possible to reduce the effect of wavelength dispersion caused by refraction.

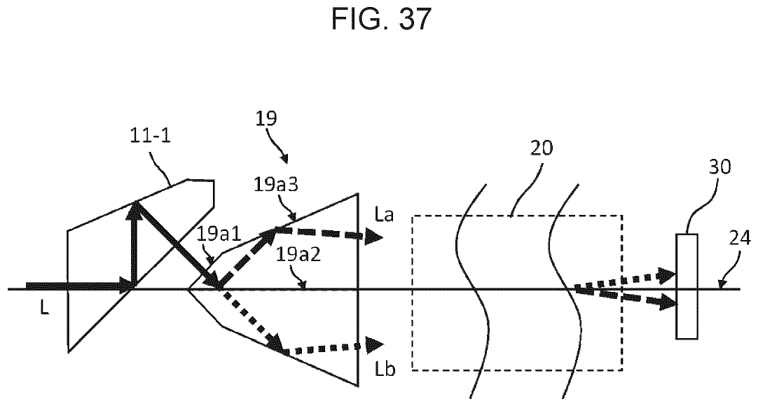

FIG. 36 is a diagram schematically illustrating an example of the paths of the light beam L, the first light beam La, and the second light beam Lb within the optical component 10A according to Modification 2. As illustrated in FIG. 36, the light beam L incident on the optical path adjusting element 11-1 in the direction q is reflected inside and then emitted to the outside. The traveling direction of the light beam L emitted to the outside from the optical path adjusting element 11-1 is φ+2ρ+2μ.

The light beam L is incident on the face 19al and separated into the first light beam La and the second light beam Lb by the face 19a2. The first light beam La is totally reflected by the face 19a3 and emitted to the outside from the face 19a4. The second light beam Lb is totally reflected by the face 19b3 and emitted to the outside from the face 19b4.

The traveling direction of the first light beam La emitted to the outside from the face 19a4 is φ+2ρ+2μ+2ξ+2τ. The traveling direction of the second light beam Lb emitted to the outside from the face 19b4 is −φ−2ρ−2μ−2ξ−2π. Therefore, as in Embodiment 1 and Modification 1 thereof, the first light beam La and the second light beam Lb are in a mirror-image symmetry relationship with respect to the plane 24.

When ρ is about π/4 and μ is about 5π/8, φ+2μ+2μ is about −π/4 if φ is approximately zero. Furthermore, when ξ is about π/4 and τ is about (7/8)π, the traveling directions of the first light beam La and the second light beam Lb become nearly zero. However, as in Embodiment 1 and Modification 1 thereof, the traveling directions of the first light beam La and the second light beam Lb do not have to be completely zero. By adjusting any one of ρ, μ, ξ, and τ, an angular difference can be imparted to the traveling directions of the first light beam La and the second light beam Lb even if φ is nearly zero. When the first light beam La and the second light beam Lb travel while approaching each other, the first and second images that are mirror-image symmetric with respect to the plane 24 can be formed at different positions on the image sensor 30.

FIG. 37 is a diagram schematically illustrating an example of the paths of the light beam L, the first light beam La, and the second light beam Lb in Modification 2 of the imaging device. As in Embodiment 1 and Modification 1 thereof, the first and second images can be formed on the image sensor 30 by combining the dichroic prism 19 and the optical path adjusting element 11-1 with the imaging optical system 20, as illustrated in FIG. 37.

In Modification 2, the incident angle of the light beam L on the face 19a2 is π/4. Therefore, compared to Embodiment 1 in which the incident angle is as steep as π/3, the dielectric multilayer film constituting the face 19a2 is easier to design and manufacture.

Furthermore, in Modification 2, the face 19al as the incident surface on which the light beam L is incident is separated from the face 19a3 as the reflecting surface that reflects the first light beam La and emits it to the outside of the dichroic prism 19. Therefore, as for the face 19a1, there is no need to consider the total reflection conditions for the first light beam La, thus easing restrictions on the material of the dichroic prism 19.

Furthermore, in Modification 2, the dichroic prism 19 is formed of a single optical element. Therefore, compared to Modification 1 in which the optical element group 14 includes a plurality of optical elements, the dichroic prism 19 is easier to assemble and adjust.

### Embodiment 2

In Embodiment 1 and Modifications 1 and 2 thereof, the imaging device obtains two images of the subject in different wavelength ranges as an example of two images having different optical characteristics. As another example of two images having different optical characteristics, the imaging device may obtain two images of the subject having different polarization states.

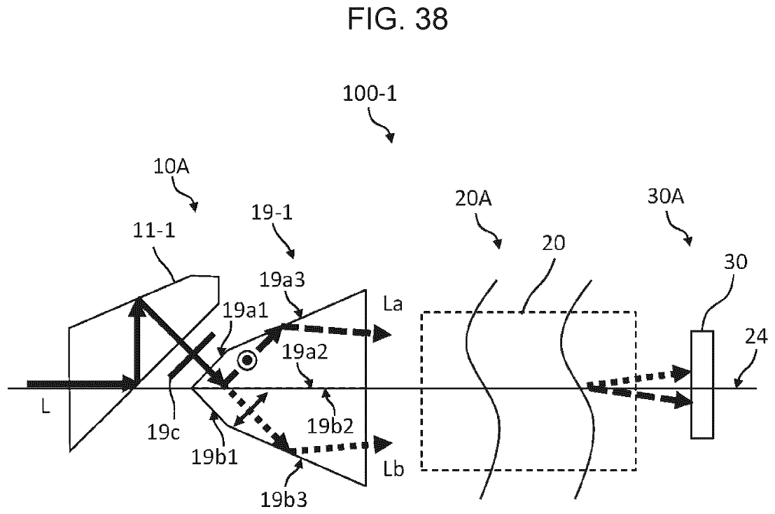

FIG. 38 is a diagram schematically illustrating a specific configuration of an imaging device according to exemplary Embodiment 2 of the present disclosure. An imaging device 100-1 illustrated in FIG. 38 obtains two images of a subject 110 having different polarization states. As illustrated in FIG. 38, the imaging device 100-1 includes an optical component 10A, a lens unit 20A, and a camera 30A.

The optical component 10A includes an optical path adjusting element 11-1, a prism 19-1, and a phase plate 19c. However, the phase plate 19c is not an essential component. The lens unit 20A includes an imaging optical system 20. The camera 30A includes an image sensor 30. The optical path adjusting element 11-1, the imaging optical system 20, and the image sensor 30 are as described above. The lens unit 20A may further include a lens housing 22 that houses the imaging optical system 20, as illustrated in FIG. 3. The camera 30A may further include a camera housing 32 that houses the image sensor 30, as illustrated in FIG. 3. In this specification, the prism 19-1 is also referred to as a “first optical element”.

The prism 19-1 illustrated in FIG. 38 differs from the dichroic prism 19 illustrated in FIG. 34 in that one or both of faces 19a2 and 19b2 are polarizing beam splitter surfaces that reflect a first light beam La in a first polarization state and transmit a second light beam Lb in a second polarization state, of a light beam L incident from a direction in a specific range. The first and second polarization states are different from each other. The polarizing beam splitter surface reflects light having an electric field vector parallel to the polarizing beam splitter surface and transmits light having an electric field vector perpendicular to the polarizing beam splitter surface. In the example illustrated in FIG. 38, the first polarization state is S-polarized light and the second polarization state is P-polarized light. The polarizing beam splitter surface is disposed on the plane 24.

When light is incident on the polarizing beam splitter surface from a direction of about −π/4, a film having the above function is easily designed. Therefore, the prism 19-1 has a shape illustrated in FIG. 38 so that the light beam L is incident on one or both of the faces 19a2 and 19b2 from the direction of about −π/4, and the light beam L is incident almost perpendicularly on the face 19a1.

However, the polarization states of the incident light and the emitted light may be changed by passing through the optical path adjusting element 11-1. For example, linearly polarized light incident on the optical path adjusting element 11-1 may be changed to elliptically polarized light and then emitted. This is because reflection inside the optical path adjusting element 11-1 can change the polarization state.

The phase plate 19c compensates for the change in the polarization state caused by the optical path adjusting element 11-1. As a result, the linearly polarized light incident on the optical path adjusting element 11-1 can be made incident on the prism 19-1 as linearly polarized light.

The first light beam La and the second light beam Lb emitted from the prism 19-1 are symmetrical with respect to the plane 24. Therefore, two images having different polarization states can be formed simultaneously in mirror-image symmetry on the imaging surface of the image sensor 30. This makes it possible to obtain two images having different polarization states even if the image sensor 30 has sensitivity of each pixel with no polarization dependency. The image sensor 30 may be, for example, a normal image sensor, or a hyperspectral image sensor capable of obtaining spectral information of light.

Note that even if the light beam L incident on the prism 19-1 is linearly polarized light, the first light beam La and the second light beam Lb emitted from the prism 19-1 may become elliptically polarized light by passing through the prism 19-1. Even in such a case, in the image sensor 30 in which the sensitivity of each pixel has no polarization dependency, the polarization state of the imaging result is determined only by the imaging position on the image sensor 30. This eliminates the need to compensate for the change in polarization state caused by the prism 19-1.

The imaging device 100-1 according to Embodiment 2 can thus obtain two images having different polarization states suitable for evaluating the subject 110. As described in Embodiment 1, the imaging device 100-1 can obtain these images in a natural orientation with a simple configuration.

### Modification of Imaging Device 100-1 According to Embodiment 2

Modifications 1 to 3 of the imaging device 100-1 according to Embodiment 2 will be described below.

### Modification 1

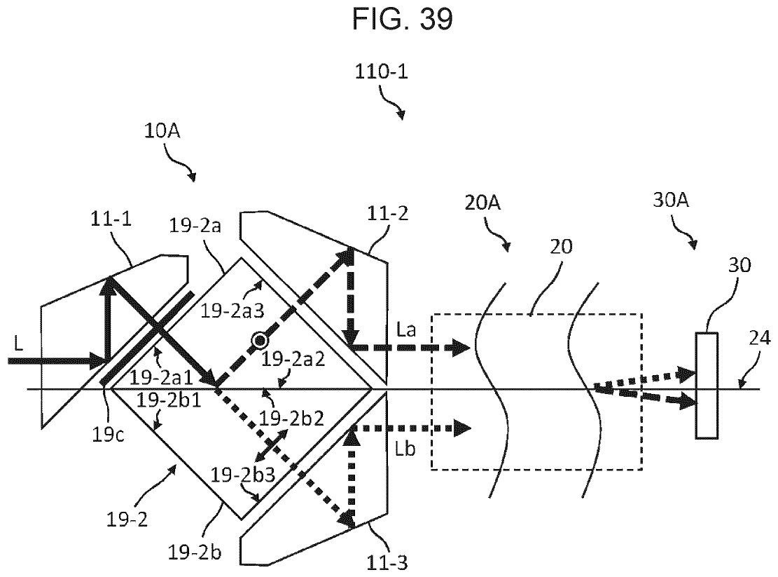

FIG. 39 is a diagram schematically illustrating a configuration of Modification 1 of the imaging device 100-1 according to Embodiment 2. An imaging device 110-1 illustrated in FIG. 39 differs from the imaging device 100-1 illustrated in FIG. 38 in the configuration of the optical component 10A. The optical component 10A illustrated in FIG. 39 includes an optical path adjusting element 11-1, an optical path adjusting element 11-2, an optical path adjusting element 11-3, a beam splitter cube 19-2, and a phase plate 19c. However, the phase plate 19c is not an essential component. As the optical path adjusting elements 11-1, 11-2, and 11-3, commercially available half pentaprisms can be used. As the beam splitter cube 19-2, a commercially available polarizing beam splitter cube can be used. In this specification, the beam splitter cube 19-2, the optical path adjusting element 11-2, and the optical path adjusting element 11-3 are also collectively referred to as a “first optical element”.

The beam splitter cube 19-2 includes a prism 19-2a and a prism 19-2b. A rectangular parallelepiped is formed by combining the two, each of which is a right-angle prism.

The prism 19-2a has a face 19-2a1, a face 19-2a2, and a face 19-2a3. The face 19-2a1 and the face 19-2a3 form a right angle. The face 19-2a2 forms an acute angle with the face 19-2a1 and also forms an acute angle with the face 19-2a3.

Similarly, the prism 19-2b has a face 19-2b1, a face 19-2b2, and a face 19-2b3. The face 19-2b1 and the face 19-2b3 form a right angle. The face 19-2b2 forms an acute angle with the face 19-2b1 and also forms an acute angle with the face 19-2b3.

The beam splitter cube 19-2 has a polarizing beam splitter surface at the interface between the face 19-2a2 and the face 19-2b2. The polarizing beam splitter surface is as described above. The first light beam La of the light beam L from the subject 110 is incident on the face 19-2a1, reflected by the face 19-2a2, and emitted from the face 19-2a3. The second light beam Lb of the light beam L from the subject 110 is incident on the face 19-2a1, passes through the faces 19-2a2 and 19-2b2, and is emitted from the face 19-2b3. The phase plate 19c that compensates for a change in the polarization state caused by the optical path adjusting element 11-1 may be disposed between the optical path adjusting element 11-1 and the beam splitter cube 19-2.

The optical path adjusting element 11-1 changes the traveling direction of the light beam L from the subject 110, which is incident from a direction substantially parallel to the optical axis of the imaging optical system 20, by about −π/4, and makes it incident on the beam splitter cube 19-2. The beam splitter cube 19-2 emits the first light beam La of the light beam L from the subject 110 in a direction in which the traveling direction thereof is inverted with respect to the polarizing beam splitter surface, and makes it incident on the optical path adjusting element 11-2. The beam splitter cube 19-2 emits the second light beam Lb of the light beam L from the subject 110 in the same direction as the traveling direction at the time of incidence, and makes it incident on the optical path adjusting element 11-3.

The optical path adjusting element 11-2 changes the traveling direction of the incident first light beam La by about −π/4 and makes it incident on the imaging optical system 20. The optical path adjusting element 11-3 changes the traveling direction of the incident second light beam Lb by about π/4 and makes it incident on the imaging optical system 20. By arranging the polarizing beam splitter surface on the plane 24, the first light beam La emitted from the optical path adjusting element 11-2 and the second light beam Lb emitted from the optical path adjusting element 11-3 are symmetrical with respect to the plane 24. Therefore, two images having different optical states can be formed in mirror-image symmetry. This makes it possible to obtain two images having different polarization states even if the image sensor 30 has sensitivity of each pixel with no polarization dependency.

### Modification 2

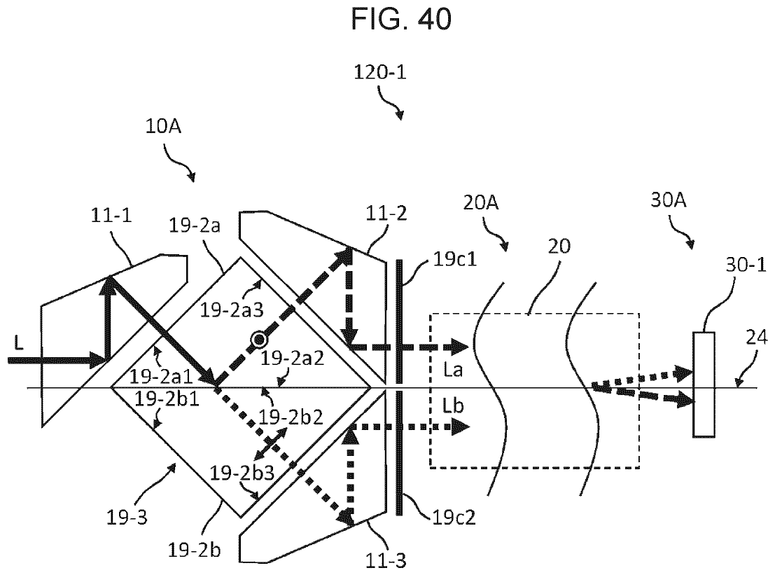

FIG. 40 is a diagram schematically illustrating a configuration of Modification 2 of the imaging device 100-1 according to Embodiment 2. An imaging device 120-1 illustrated in FIG. 40 enables full Stokes imaging. The imaging device 120-1 illustrated in FIG. 40 differs from the imaging device 110-1 illustrated in FIG. 39 in the configuration of the optical component 10A and the configuration of the camera 30A.

The optical component 10A illustrated in FIG. 40 includes a beam splitter cube 19-3, which is a polarization-independent beam splitter cube, instead of the beam splitter cube 19-2 illustrated in FIG. 39. The optical component 10A illustrated in FIG. 40 further includes a phase plate 19cl disposed on the light emission side of an optical path adjusting element 11-2 and a phase plate 19c2 disposed on the light emission side of an optical path adjusting element 11-3, instead of the phase plate 19c illustrated in FIG. 39. The camera 30A illustrated in FIG. 40 includes a polarization image sensor 30-1 capable of measuring linearly polarized light components, instead of the image sensor 30 illustrated in FIG. 39. In this specification, the beam splitter cube 19-3, the optical path adjusting element 11-2, the optical path adjusting element 11-3, the phase plate 19c1, and the phase plate 19c2 are also collectively referred to as a “first optical element”.

The beam splitter cube 19-3 is the same as the beam splitter cube 19-2, except that one or both of the faces 19-2a2 and 19-2b2 are semi-transmissive films. The semi-transmissive film reflects half of the light beam L incident from a direction of about π/4 as a first light beam La and transmits the other half as a second light beam Lb, regardless of the polarization state. The semi-transmissive film is disposed on the plane 24.

The optical path adjusting element 11-1 changes the traveling direction of the light beam L from the subject 110, which is incident from a direction almost parallel to the optical axis of the imaging optical system 20, by about −π/4, and makes it incident on the beam splitter cube 19-3. The beam splitter cube 19-3 emits the first light beam La of the light beam L from the subject 110 in a direction in which the traveling direction thereof is inverted with respect to the polarizing beam splitter surface, and makes it incident on the optical path adjusting element 11-2. The beam splitter cube 19-3 emits the second light beam Lb of the light beam L from the subject 110 in the same direction as the traveling direction at the time of incidence, and makes it incident on the optical path adjusting element 11-3.

The optical path adjusting element 11-2 changes the traveling direction of the incident first light beam La by about −π/4, and makes it incident on the imaging optical system 20 through the phase plate 19c1. The optical path adjusting element 11-3 changes the traveling direction of the incident second light beam Lb by about π/4, and makes it incident on the imaging optical system 20 through the phase plate 19c2.

The first light beam La incident on the imaging optical system 20 through the phase plate 19c1 has its polarization state changed by passing through the optical path adjusting element 11-1, reflection at the beam splitter cube 19-3, and passing through the optical path adjusting element 11-2 before entering the phase plate 19c1. The second light beam Lb incident on the imaging optical system 20 through the phase plate 19c2 has its polarization state changed by passing through the optical path adjusting element 11-1, passing through the beam splitter cube 19-3, and passing through the optical path adjusting element 11-3 before entering the phase plate 19c2.

The phase plate 19cl then compensates for the above change in the polarization state of the first light beam La, and restores the polarization state of the first light beam La to the polarization state at the subject 110. On the other hand, the phase plate 19c2 compensates for the above change in the polarization state of the second light beam Lb, and restores the polarization state of the second light beam Lb to the polarization state at the subject 110, and further imparts a polarization phase of π/4 to the second light beam Lb.

In this case, the image of the first light beam La formed by the imaging optical system 20 is an image in the polarization state at the subject 110 as it is. The image of the second light beam Lb formed by the imaging optical system 20 is an image with a phase shift of π/4 from the polarization state at the subject 110. By taking these two images using the polarization image sensor 30-1, all components of the Stokes parameters can be obtained in one imaging. Information on the elliptical polarization of the subject 110 can be obtained from all components of the Stokes parameters. Obtaining all components of the Stokes parameters is effective in product inspection and material analysis, for example.

### Modification 3

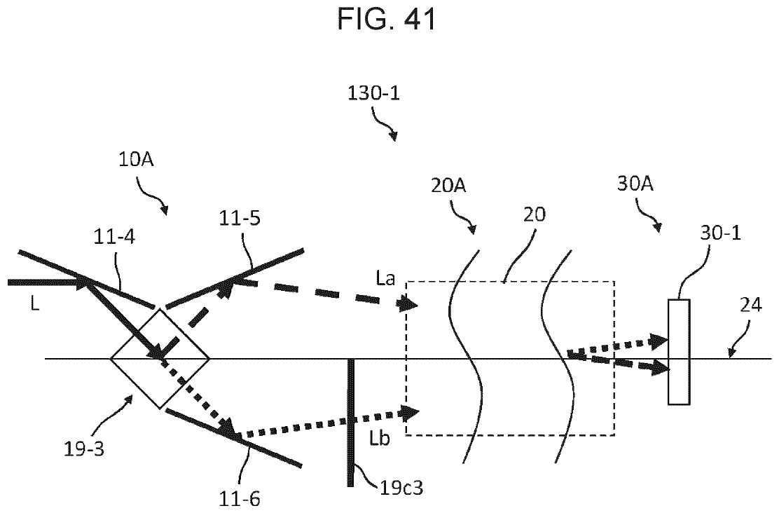

FIG. 41 is a diagram schematically illustrating a configuration of Modification 3 of the imaging device 100-1 according to Embodiment 2. An imaging device 130-1 illustrated in FIG. 41 differs from the imaging device 120-1 illustrated in FIG. 40 in the configuration of the optical component 10A.

The optical component 10A illustrated in FIG. 41 includes optical path adjusting elements 11-4, 11-5, and 11-6, each of which is a metal mirror, instead of the optical path adjusting elements 11-1, 11-2, and 11-3, which are half pentaprisms illustrated in FIG. 40. The polarization state is changed by reflection on the dielectric interface, not by reflection on the metal interface. Therefore, the optical component 10A illustrated in FIG. 41 has no need to compensate for the change in the polarization state.

The optical component 10A illustrated in FIG. 41 further includes a phase plate 19c3 on the light emission side of the optical path adjusting element 11-6. The phase plate 19c3 changes the polarization phase of the second light beam Lb by π/4.

In this specification, the beam splitter cube 19-3, the optical path adjusting element 11-5, the optical path adjusting element 11-6, and the phase plate 19c3 are also collectively referred to as a “first optical element”, and the optical path adjusting element 11-4 is also referred to as a “second optical element”.

As in Modification 2, all components of the Stokes parameters can also be obtained in one imaging in Modification 3.

### APPLICATION EXAMPLE

Application examples 1 to 3 of the imaging device 100 according to Embodiment 1 will be described below.

### Application Example 1: Temperature Measurement

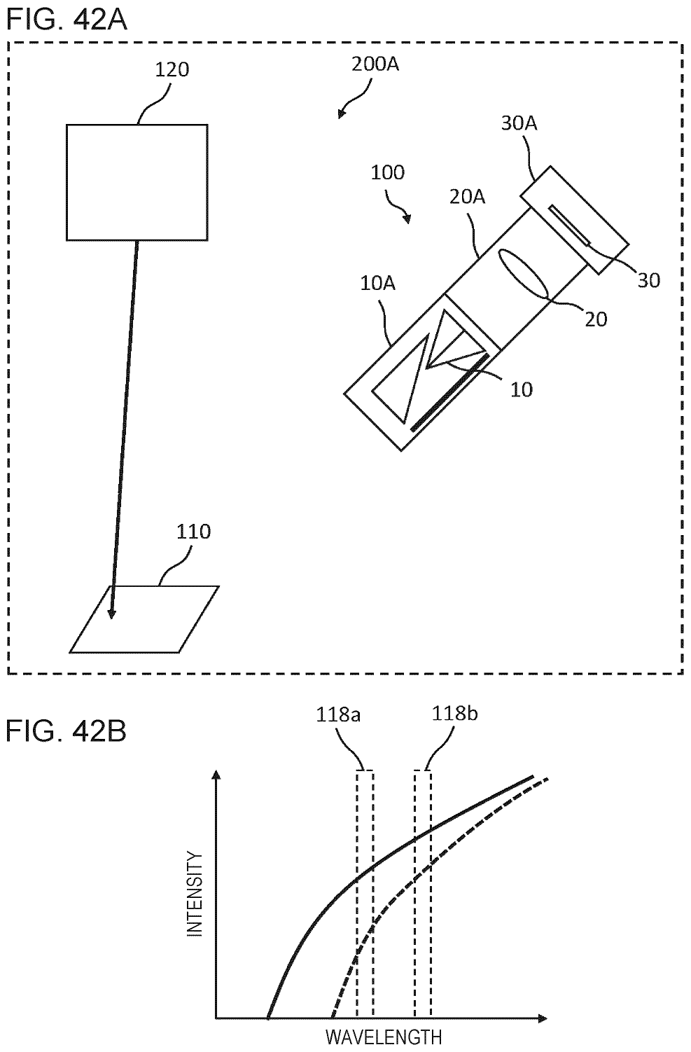

With reference to FIGS. 42A and 42B, description will be given of temperature measurement using the imaging device 100 according to Embodiment 1. FIG. 42A is a diagram schematically illustrating an example of a measurement system including the imaging device 100 according to Embodiment 1. A measurement system 200A illustrated in FIG. 42A measures the temperature of the subject 110.

As illustrated in FIG. 42A, the measurement system 200A includes the imaging device 100 that performs imaging of the subject 110, and a heating device 120 that heats the subject 110. The heating device 120 may locally heat a portion of the subject 110, or may heat the entire subject 110. The heating device 120 may be, for example, a laser device that locally heats the subject 110 with a laser beam represented by a thick line, as illustrated in FIG. 42A. Alternatively, the heating device 120 may be a heating lamp or resistance heater that heats the entire subject 110.

As the first and second wavelength ranges, wavelength ranges that provide a sufficiently high thermal radiation intensity in the temperature range for monitoring the subject 110 are selected. When the temperature range for monitoring is higher than or equal to 200° C. and lower than or equal to 500° C. and the image sensor 30 is formed of InGaAs or quantum dots, two different wavelength ranges can be selected from a wavelength range of greater than or equal to 1.3 μm to less than or equal to 1.6 μm, as the first and second wavelength ranges.

As the first and second wavelength ranges, two different wavelength ranges in which the subject 110 has almost the same emissivity, for example, can be selected. When the subject 110 is made of a metal material or ceramic, the subject 110 often does not have characteristic absorption in the wavelength range of greater than or equal to 1.3 μm and less than or equal to 1.6 μm. When the subject 110 is made of an organic material such as resin, the subject 110 may have resonant absorption in the wavelength range of greater than or equal to 1.3 μm and less than or equal to 1.6 μm. In the wavelength range in which resonant absorption occurs, the emissivity differs significantly from the surrounding wavelength ranges. Therefore, the wavelength range in which strong resonant absorption occurs may be avoided as the first and second wavelength ranges.

The measurement system 200A may further include an optical element as an auxiliary optical element that attenuates or blocks light having a wavelength other than the first and second wavelength ranges. For example, when the subject 110 is placed under illumination with visible light and the image sensor 30 is sensitive to the visible light, the measurement system 200A may further include an optical element that blocks the visible light.

When the subject 110 is heated by the heating device 120, the temperature of the subject 110 rises, causing thermal radiation according to the temperature and emissivity. FIG. 42B is a diagram schematically illustrating an example of the spectrum of thermal radiation from the subject 110. The solid line illustrated in FIG. 42B represents a spectrum in a high-temperature region of the subject 110, and the dashed line illustrated in FIG. 42B represents a spectrum in a low-temperature region of the subject 110. In the high-temperature region, thermal radiation occurs with a high intensity at any wavelength, compared to the low-temperature region. However, the intensity ratio is larger on the short-wavelength side and smaller on the long-wavelength side. When two different wavelength ranges with almost the same emissivity are selected as a first wavelength range 118a and a second wavelength range 118b, a radiation intensity ratio of the first and second wavelength ranges depends on temperature but does not depend on emissivity.

Therefore, two different wavelength ranges with sufficient radiation intensity for imaging and almost the same emissivity are selected as the first and second wavelength ranges 118a and 118b. The first and second wavelength ranges 118a and 118b have almost the same width.

In the measurement system 200A, a first image 110a and a second image 110b of the subject 110 are simultaneously obtained, and the radiation intensity ratio of the first and second wavelength ranges 118a and 118b is calculated. This makes it possible to determine the temperature distribution of the subject 110 regardless of the emissivity.

In the measurement system 200A, light having a wavelength included in the first wavelength range and light having a wavelength included in the second wavelength range, which are generated by thermal radiation, are imaged. The measurement system 200A does not include an illuminating device that emits light having wavelengths included in these wavelength ranges. Alternatively, such an illuminating device is not used during temperature measurement. When there is ambient light that includes light having a wavelength included in the first wavelength range and light having a wavelength included in the second wavelength range, such as sunlight, the subject 110 and the imaging device 100 may be surrounded by a light shielding body, so that such ambient light does not enter the subject 110.

### Application Example 2: Measurement of Fluorescence Luminous Efficiency

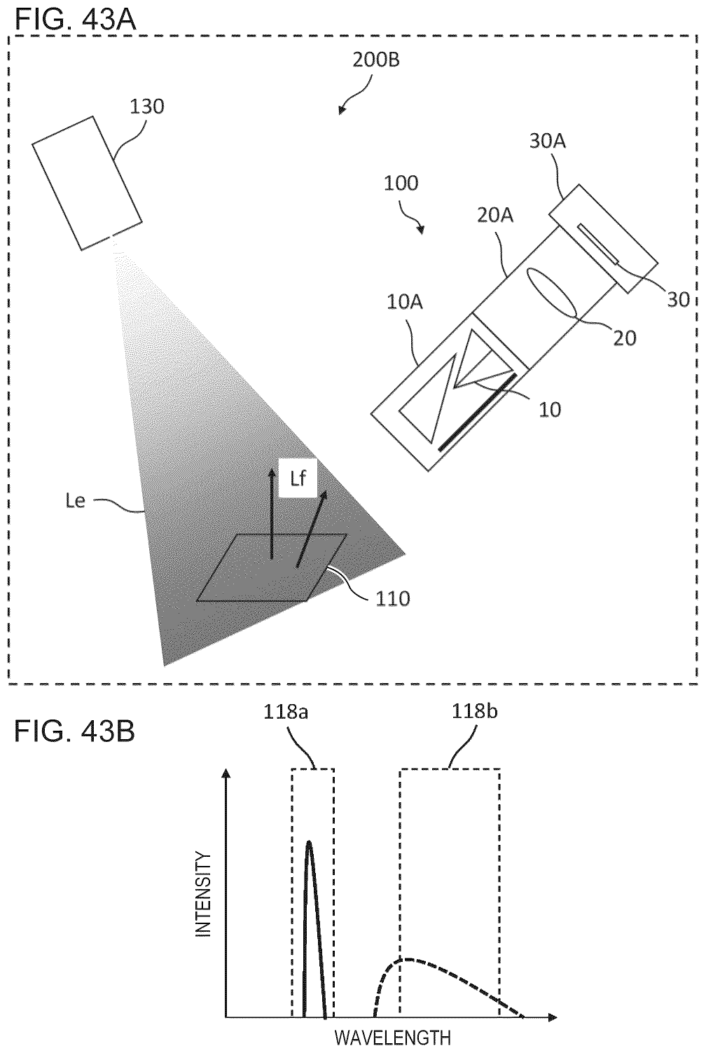

With reference to FIGS. 43A and 43B, description will be given of measurement of fluorescence luminous efficiency using the imaging device 100 according to Embodiment 1. FIG. 43A is a diagram schematically illustrating another example of a measurement system including the imaging device 100 according to Embodiment 1. A measurement system 200B illustrated in FIG. 43A measures the fluorescence luminous efficiency of the subject 110. The subject 110 contains a fluorescent dye that absorbs light having a wavelength included in a first wavelength range and emits light having a wavelength included in a second wavelength range. In this case, the second wavelength range is on the longer wavelength side than the first wavelength range.

The measurement system 200B includes an imaging device 100 that performs imaging of the subject 110, and an illuminating device 130 that emits excitation light Le as illuminating light for irradiating the subject 110. When the subject 110 is irradiated with the excitation light Le, fluorescence Lf is emitted from an area of the subject 110 that has a non-zero luminous efficiency.

As the first wavelength range, a wavelength range that includes a wavelength range of the excitation light Le and does not substantially include a wavelength range of the fluorescence Lf is selected. As the second wavelength range, a wavelength range that includes the wavelength range of the fluorescence Lf and does not substantially include the wavelength range of the excitation light Le is selected. The excitation light Le includes light having a wavelength included in the first wavelength range, and does not include light having a wavelength included in the second wavelength range.

FIG. 43B is a diagram schematically illustrating an example of spectra of the excitation light Le and the fluorescence Lf. The solid line illustrated in FIG. 43B represents the spectrum of the excitation light Le, and the dashed line illustrated in FIG. 43B represents the spectrum of the fluorescence Lf. The spectrum of the excitation light Le has a relatively narrow width, whereas the spectrum of the fluorescence Lf has a relatively wide width. Therefore, a first wavelength range 118a corresponding to the excitation light Le has a relatively narrow width, whereas a second wavelength range 118b corresponding to the fluorescence Lf has a relatively wide width.

The intensity of the excitation light Le on the subject 110 is distributed two-dimensionally depending on the optical characteristics of the illuminating device 130 and the shape of the subject 110. The intensity of the fluorescence Lf is proportional to the intensity and luminous efficiency of the excitation light Le.

When the subject 110 is excited by the excitation light Le emitted from the illuminating device 130, the intensity of the excitation light Le is obtained as a first image 110a, the intensity of the fluorescence Lf is obtained as a second image 110b, and the intensity ratio thereof is calculated. This makes it possible to visualize the distribution of luminous efficiency of the subject 110, regardless of the intensity distribution of the excitation light Le.

The distribution of luminous efficiency can be related to, for example, a staining concentration when the subject 110 is stained with a fluorescent material. If this fluorescence staining is performed by a so-called antigen-antibody staining method, the antigen concentration can be visualized.

Alternatively, the distribution of luminous efficiency can be related to, for example, a distribution of a quencher. The more densely the quencher is distributed in an area, the lower the luminous efficiency. The measurement system 200B can thus obtain information on the concentration of the quencher.

### Application Example 3: Visualization of Material Distribution

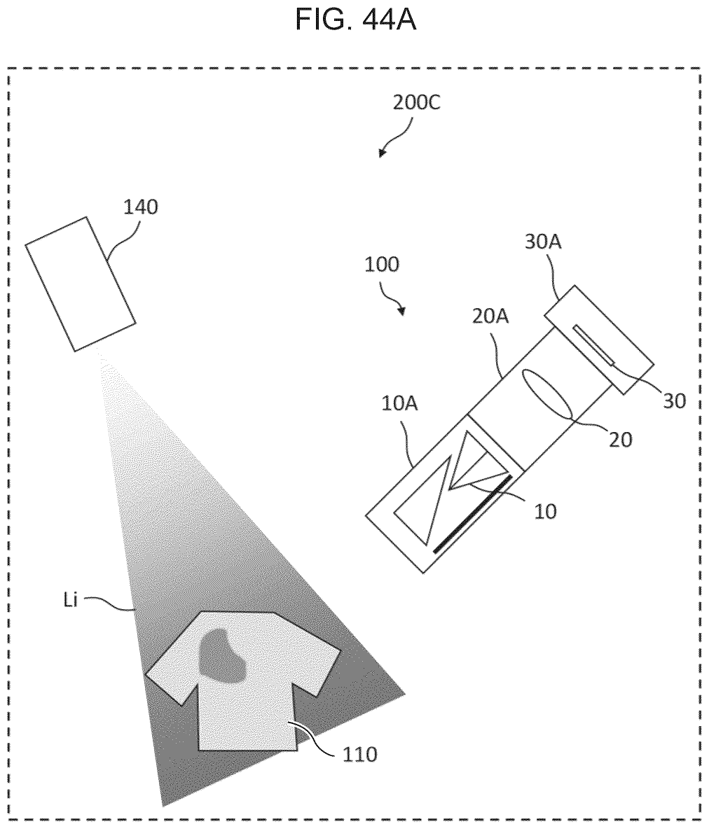

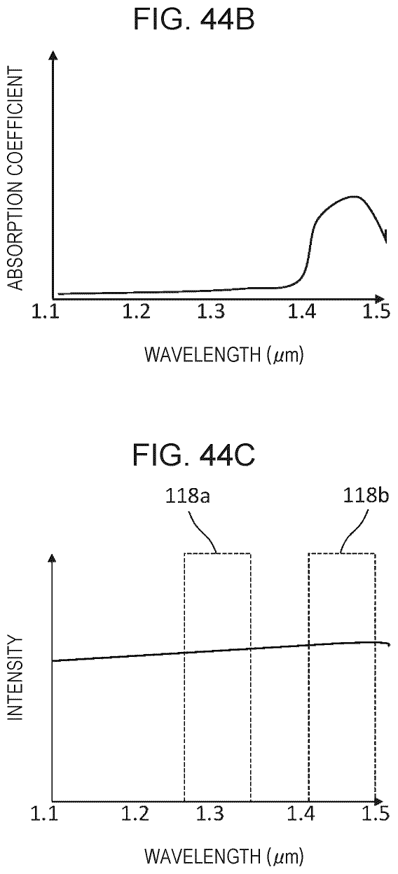

With reference to FIGS. 44A to 44C, description will be given of visualization of a material distribution using the imaging device 100 according to Embodiment 1. FIG. 44A is a diagram schematically illustrating yet another example of a measurement system including the imaging device 100 according to Embodiment 1. A measurement system 200C illustrated in FIG. 44A visualizes the distribution of a specific material in the subject 110.

The measurement system 200C includes the imaging device 100 that performs imaging of the subject 110, and an illuminating device 140 that emits illuminating light Li for irradiating the subject 110. The illuminating light Li includes light having a wavelength included in a first wavelength range and light having a wavelength included in a second wavelength range. Note that if ambient light includes light having a wavelength included in the first wavelength range and light having a wavelength included in the second wavelength range, the illuminating device 140 may be omitted.

The subject 110 includes at least two types of materials, a first material and a second material. In a case where two types of materials are first and second materials, it is assumed that the first material has approximately equal reflectance in the first and second wavelength ranges, and the second material has significantly different reflectances in the first and second wavelength ranges. The absolute value of a difference in reflectance between the first and second wavelength ranges of the first material may be, for example, less than or equal to 5%. The absolute value of a difference in reflectance between the first and second wavelength ranges of the second material may be, for example, more than or equal to 10%. The ratio of the reflectance between the first and second wavelength ranges of the first material is different from the ratio of the reflectance between the first and second wavelength ranges of the second material.

A first image 110a and a second image 110b of the subject 110 illuminated with the illuminating light Li are obtained by the imaging device 100, and the intensity ratio thereof is calculated. In a region of the subject 110 where only the first material is distributed, the intensity ratio is close to 1, whereas the intensity ratio deviates from 1 in a region where the second material is distributed in large amounts. Therefore, the distribution of the second material in the subject 110 can be visualized by the intensity ratio of the two.

One example where this material visualization is effective is the visualization of wet clothing. FIG. 44B is a diagram schematically illustrating an example of the absorption spectrum of water. The absorption and reflectance spectra of materials differ from material to material. When the material is water, the absorption coefficient is small in a wavelength range of less than or equal to 1.35 μm, as illustrated in FIG. 44B. On the other hand, the absorption coefficient is large in a wavelength range of greater than or equal to 1.4 μm and less than or equal to 1.5 μm. Many fibers used in clothing do not show as strong wavelength dependency of the absorption coefficient as water in these wavelength ranges.

Therefore, when a piece of clothing includes a first region that is wet with water and a second region that is not wet with water, the reflectance does not differ significantly between the first and second regions in the wavelength range of less than or equal to 1.35 μm where the absorption coefficient of water is small. On the other hand, the reflectance in the first region is significantly lower than the reflectance in the second region in the wavelength range of greater than or equal to 1.4 μm and less than or equal to 1.5 μm where the absorption coefficient of water is large.

Therefore, the wavelength range where the absorption coefficient of water is small is selected as the first wavelength range, whereas the wavelength range where the absorption coefficient of water is large is selected as the second wavelength range. The illuminating device 140 is also used, which emits light in the first and second wavelength ranges as the illuminating light Li. FIG. 44C is a diagram schematically illustrating an example of the spectrum of the illuminating light Li. As illustrated in FIG. 44C, the illuminating light Li has an approximately equal intensity in the first and second wavelength ranges 118a and 118b. The first and second wavelength ranges 118a and 118b have almost the same width.

The intensity of the illuminating light Li on the subject 110 is distributed two-dimensionally depending on the optical characteristics of the illuminating device 140 and the shape of the subject 110. By adjusting the illuminating light Li, it is possible to achieve corresponding intensity distributions, on the subject 110, of the light having wavelengths included in the first and second wavelength ranges 118a and 118b included in the illuminating light Li.

The first image 110a and the second image 110b of the subject 110 illuminated with the illuminating light Li are obtained by the imaging device 100, and the intensity ratio thereof is calculated. The intensity ratio does not depend on the intensity distribution of the illuminating light Li, but on the ratio of the reflectance of the subject 110 between the first and second wavelength ranges 118a and 118b. This makes it possible to visualize the region of the subject 110 with a large absorption coefficient, that is, a wet region.

### APPENDIX

The following technologies are disclosed based on the above description of the embodiments.

### Technology 1

An imaging device including:
a first optical element that separates a light beam from a subject into a first light beam and a second light beam having optical characteristics different from optical characteristics of the first light beam;
an imaging optical system on which the first light beam and the second light beam are incident at different angles from each other, the imaging optical system forming a first image by imaging the first light beam and forming a second image by imaging the second light beam; and
an image sensor including an imaging surface, in which
the first image and the second image are formed at different positions on the imaging surface, and
the first image and the second image are formed symmetrically on the imaging surface with respect to a plane that intersects the imaging surface.

This imaging device can obtain two images with different optical characteristics from each other suitable for evaluating the subject with a simple configuration.

### Technology 2

The imaging device according to technology 1, further including:
a connection structure for fixing a positional relationship between the first optical element and the imaging optical system.

In this imaging device, a dichroic prism and the imaging optical system can be fixed in a desired positional relationship, more specifically, in a desired angular relationship.

### Technology 3

The imaging device according to technology 1 or 2, further including:
a lens housing; and
a camera housing, in which
the lens housing includes the imaging optical system,
the camera housing includes the image sensor, and
the lens housing and the camera housing are attachable to and detachable from the imaging device.

In this imaging device, a general lens housing and a general camera housing can be appropriately combined.

### Technology 4

The imaging device according to any one of technologies 1 to 3, in which
the first image and the second image formed on the imaging surface are each smaller than the subject.

This imaging device can reduce, even in the above case, aberration caused when a light beam having a spread angle passes through the optical element.

### Technology 5

The imaging device according to any one of technologies 1 to 4, further including:
a second optical element that changes a direction of the light beam from the subject and that makes the light beam incident on the first optical element.

In this imaging device, the imaging optical system can be oriented in a natural direction.

### Technology 6

The imaging device according to any one of technologies 1 to 5, further including:
a light shielding body that blocks an unintended light beam from entering the first optical element.

This imaging device can reduce stray light.

### Technology 7

The imaging device according to any one of technologies 1 to 6, in which
the first light beam has a wavelength included in a first wavelength range,
the second light beam has a wavelength included in a second wavelength range,
the first optical element includes a dichroic surface that reflects the first light beam and that transmits the second light beam, and
the dichroic surface is disposed on the plane.

This imaging device can obtain two images in different wavelength ranges from each other, as two images having different optical characteristics from each other.

### Technology 8

The imaging device according to technology 7, in which
the first optical element is a dichroic prism including the dichroic surface.

In this imaging device, a single dichroic prism can separate a light beam from the subject into a first light beam and a second light beam in different wavelength ranges from each other.

### Technology 9

The imaging device according to technology 7, in which
the first optical element includes a first mirror, a second mirror, a triangular prism, and a dichroic mirror including the dichroic surface,
the first mirror reflects the first light beam to make the first light beam incident on the triangular prism,
the second mirror reflects the second light beam to make the second light beam incident on the triangular prism, and
the triangular prism emits the first light beam and the second light beam to the outside of the triangular prism.

In this imaging device, the positions of two images in different wavelength ranges from each other on the image sensor can be adjusted by changing the orientation of the two mirrors.

### Technology 10

The imaging device according to any one of technologies 1 to 6, in which
the first light beam has a first polarization state,
the second light beam has a second polarization state,
the first optical element includes a polarizing beam splitter surface that reflects the first light beam and that transmits the second light beam, and
the polarizing beam splitter surface is disposed on the plane.

This imaging device can obtain two images having different polarization states from each other, as two images having different optical characteristics from each other.

### Technology 11

The imaging device according to technology 10, in which
the first optical element is a prism including the polarizing beam splitter surface.

In this imaging device, a single prism can separate a light beam from the subject into a first light beam and a second light beam having different polarization states from each other.

### Technology 12

An optical component used in an imaging device including an imaging optical system and an image sensor, the optical component including:
a first optical element that separates a light beam from a subject into a first light beam and a second light beam having optical characteristics different from optical characteristics of the first light beam, and that emits the first light beam and the second light beam symmetrically with respect to a certain plane, in which
the first light beam and the second light beam are incident on the imaging optical system at different angles from each other,
the imaging optical system forms a first image by imaging the first light beam and forms a second image by imaging the second light beam,
the image sensor includes an imaging surface, and
the first image and the second image are formed at different positions on the imaging surface.

This optical component can obtain two images with different optical characteristics from each other suitable for evaluating the subject with a simple configuration using a general lens unit and a general camera.

### Technology 13

The optical component according to technology 12, further including:
a second optical element that changes a direction of the light beam from the subject and that makes the light beam incident on the first optical element.

This optical component can orient the imaging optical system in a natural direction.

### Technology 14

The optical component according to technology 12 or 13, further including:
a light shielding body that blocks an unintended light beam from entering the first optical element.

This optical component can reduce stray light.

### Technology 15

The optical component according to any one of technologies 12 to 14, in which
the imaging device further includes a lens housing,
the lens housing includes the imaging optical system, and
the optical component is detachable from the lens housing.

This optical component can be attached to or detached from a general lens housing.

### Technology 16

The optical component according to any one of technologies 12 to 15, in which
the first light beam has a wavelength included in a first wavelength range,
the second light beam has a wavelength included in a second wavelength range,
the first optical element includes a dichroic surface that reflects the first light beam and that transmits the second light beam, and
the dichroic surface is disposed on the plane.

This optical component can obtain two images in different wavelength ranges from each other, as two images having different optical characteristics from each other.

### Technology 17

The optical component according to technology 16, in which
the first optical element is a dichroic prism including the dichroic surface.

This optical component can separate a light beam from the subject into a first light beam and a second light beam in different wavelength ranges from each other, using a single dichroic prism.

### Technology 18

The optical component according to any one of technologies 12 to 15, in which
the first light beam has a first polarization state,
the second light beam has a second polarization state,
the first optical element includes a polarizing beam splitter surface that reflects the first light beam and that transmits the second light beam, and
the polarizing beam splitter surface is disposed on the plane.

This optical component can obtain two images having different polarization states from each other, as two images having different optical characteristics from each other.

### Technology 19

A measurement system including:
the imaging device according to any one of technologies 1 to 9; and
a heating device that heats the subject.

This measurement system can measure the temperature of the subject.

### Technology 20

A measurement system including:
the imaging device according to any one of technologies 1 to 9; and
an illuminating device that emits illuminating light for irradiating the subject.

This measurement system can measure fluorescence luminous efficiency of the subject when the subject contains a fluorescent dye.

The imaging device of the present disclosure is particularly effective in two-color thermography and fluorescence imaging. The imaging device of the present disclosure is also effective in visualizing a material distribution.

What is claimed is:

1. An imaging device comprising:
a first optical element that separates a light beam from a subject into a first light beam and a second light beam having optical characteristics different from optical characteristics of the first light beam;
an imaging optical system on which the first light beam and the second light beam are incident at different angles from each other, the imaging optical system forming a first image by imaging the first light beam and forming a second image by imaging the second light beam; and
an image sensor including an imaging surface, wherein
the first image and the second image are formed at different positions on the imaging surface, and
the first image and the second image are formed symmetrically on the imaging surface with respect to a plane that intersects the imaging surface.

2. The imaging device according to claim 1, further comprising:
a connection structure for fixing a positional relationship between the first optical element and the imaging optical system.

3. The imaging device according to claim 1, further comprising:
a lens housing; and
a camera housing, wherein
the lens housing includes the imaging optical system,
the camera housing includes the image sensor, and
the lens housing and the camera housing are attachable to and detachable from the imaging device.

4. The imaging device according to claim 1, wherein
the first image and the second image formed on the imaging surface are each smaller than the subject.

5. The imaging device according to claim 1, further comprising:
a second optical element that changes a direction of the light beam from the subject and that makes the light beam incident on the first optical element.

6. The imaging device according to claim 1, further comprising:
a light shielding body that blocks an unintended light beam from entering the first optical element.

7. The imaging device according to claim 1, wherein
the first light beam has a wavelength included in a first wavelength range,
the second light beam has a wavelength included in a second wavelength range,
the first optical element includes a dichroic surface that reflects the first light beam and that transmits the second light beam, and
the dichroic surface is disposed on the plane.

8. The imaging device according to claim 7, wherein
the first optical element is a dichroic prism including the dichroic surface.

9. The imaging device according to claim 7, wherein
the first optical element includes a first mirror, a second mirror, a triangular prism, and a dichroic mirror including the dichroic surface,
the first mirror reflects the first light beam to make the first light beam incident on the triangular prism,
the second mirror reflects the second light beam to make the second light beam incident on the triangular prism, and
the triangular prism emits the first light beam and the second light beam to an outside of the triangular prism.

10. The imaging device according to claim 1, wherein
the first light beam has a first polarization state,
the second light beam has a second polarization state,
the first optical element includes a polarizing beam splitter surface that reflects the first light beam and that transmits the second light beam, and
the polarizing beam splitter surface is disposed on the plane.

11. The imaging device according to claim 10, wherein
the first optical element is a prism including the polarizing beam splitter surface.

12. An optical component used in an imaging device including an imaging optical system and an image sensor, the optical component comprising:
a first optical element that separates a light beam from a subject into a first light beam and a second light beam having optical characteristics different from optical characteristics of the first light beam, and that emits the first light beam and the second light beam symmetrically with respect to a certain plane, wherein
the first light beam and the second light beam are incident on the imaging optical system at different angles from each other,
the imaging optical system forms a first image by imaging the first light beam and forms a second image by imaging the second light beam,
the image sensor includes an imaging surface, and
the first image and the second image are formed at different positions on the imaging surface.

13. The optical component according to claim 12, further comprising:
a second optical element that changes a direction of the light beam from the subject and that makes the light beam incident on the first optical element.

14. The optical component according to claim 12, further comprising:
a light shielding body that blocks an unintended light beam from entering the first optical element.

15. The optical component according to claim 12, wherein
the imaging device further includes a lens housing,
the lens housing includes the imaging optical system, and
the optical component is attachable to and detachable from the lens housing.

16. The optical component according to claim 12, wherein
the first light beam has a wavelength included in a first wavelength range,
the second light beam has a wavelength included in a second wavelength range,
the first optical element includes a dichroic surface that reflects the first light beam and that transmits the second light beam, and
the dichroic surface is disposed on the plane.

17. The optical component according to claim 16, wherein
the first optical element is a dichroic prism including the dichroic surface.

18. The optical component according to claim 12, wherein
the first light beam has a first polarization state,
the second light beam has a second polarization state,
the first optical element includes a polarizing beam splitter surface that reflects the first light beam and that transmits the second light beam, and
the polarizing beam splitter surface is disposed on the plane.

19. A measurement system comprising:
the imaging device according to claim 1; and
a heating device that heats the subject.

20. A measurement system comprising:
the imaging device according to claim 1; and
an illuminating device that emits illuminating light for irradiating the subject.
